(19) United States
(12) Patent Application Publication
(10) Pub. No.: US 2025/0188046 A1
(43) Pub. Date: Jun. 12, 2025

(54) ULTRAFAST FLOW SYNTHESIS OF FUNCTIONALIZED SULFONYL FLUORIDES AND SUBSEQUENT SUFEX CONNECTIONS VIA LITHIATED CHEMISTRY

(71) Applicant: POSTECH RESEARCH AND BUSINESS DEVELOPMENT FOUNDATION, Pohang-si (KR)

(72) Inventors: Dong-Pyo KIM, Pohang-si (KR); Ji-Ho KANG, Pohang-si (KR)

(21) Appl. No.: 18/911,471

(22) Filed: Oct. 10, 2024

(30) Foreign Application Priority Data
Dec. 8, 2023 (KR) 10-2023-0177851
Sep. 6, 2024 (KR) 10-2024-0121867

(51) Int. Cl. C07D 275/06; C07C 315/04; C07D 261/20

(52) U.S. Cl. CPC C07D 275/06; C07C 315/04; C07D 261/20; C07C 2601/16

(57) ABSTRACT

Disclosed are ultrafast flow synthesis of functionalized sulfonyl fluorides and subsequent SuFEx connections via lithiated chemistry. In detail, a method of synthesizing an ortho-functionalized benzenesulfonyl fluoride derivative, the method comprises (a) reacting a benzenesulfonyl fluoride derivative represented by structural formula 1 with an organolithium represented by structural formula 2, thus synthesizing an intermediate represented by structural formula 3 in reaction scheme 1 and (b) reacting the intermediate with an electrophile, thus synthesizing a compound represented by structural formula 4 or a compound represented by structural formula 4′. The present disclosure enables the synthesis of several functionalized sulfonyl fluorides within a few seconds under mild temperature conditions in addition to the chemistry of Sulfonyl Fluoride Exchange (SuFEx), which is a next-generation click chemistry, so that sulfonyl fluoride, the main reactant of the SuFEx reaction, can be efficiently secured.

### CROSS REFERENCE TO RELATED APPLICATION

The present application claims priority to Korean Patent Application No. 10-2023-0177851, filed Dec. 8, 2023, Korean Patent Application No. 10-2024-0121867, filed Sep. 6, 2024, the entire contents of which are incorporated herein for all purposes by this reference.

### BACKGROUND OF THE DISCLOSURE

### 1. Field of the Disclosure

The present disclosure relates to ultrafast flow synthesis of functionalized sulfonyl fluorides and subsequent sufex connections via lithiated chemistry.

### 2. Description of the Related Art

For the synthesis of functionalized sulfonyl fluoride, it is carried out through fluorosulfonylation with the direct introduction of sulfonyl fluoride functional groups or fluorination at sulfonyl chloride. In addition, there is a method of utilizing aryllithium intermediates with sulfonyl fluoride functional groups in batch-type reactors such as flasks.

However, in the case of fluorosulfonylation in which a sulfonyl fluoride functional group is directly introduced, a toxic reagent is used as a reactant. When the functionalized sulfonyl fluoride to be synthesized is a complex molecule in the case of fluorination in sulfonyl chloride, there is a limitation that it is necessary to proceed with fluorination immediately after synthesis due to low stability of sulfonyl chloride. When using aryl lithium intermediates, functionalized sulfonyl fluorides can be synthesized from simple molecules. However extremely low temperature conditions are required in batch reactors such as flasks, and there are problems such as low reaction selectivity, low yields, and only a limited range of functionalized sulfonyl fluorides can be synthesized.

Therefore, it is necessary to solve the above problems and research on a technology that can synthesize sulfonyl fluoride at an ultra-high speed under mild conditions.

### SUMMARY OF THE DISCLOSURE

The purpose of the present disclosure is to provide a method of synthesizing sulfonyl fluoride that can control a highly reactive ultra-short-lived aryllithium intermediate having a sulfonyl-fluoride functional group using a flow-based microreactor.

The other purpose of the present disclosure is to provide a method of synthesizing various functionalized sulfonyl fluorides by introducing various electrophiles through high site selectivity.

The other purpose of the present disclosure is to provide a method for the rapid synthesis of sulfonic compounds in one flow by successively carrying out the introduction of an unstable aryllithium intermediate.

According to one aspect of the present disclosure, there is provided a method of synthesizing an ortho-functionalized benzenesulfonyl fluoride derivative, the method comprising: (a) reacting a benzenesulfonyl fluoride derivative represented by structural formula 1 with an organolithium represented by structural formula 2, thus synthesizing an intermediate represented by structural formula 3 in reaction scheme 1; and (b) reacting the intermediate with an electrophile, thus synthesizing a compound represented by structural formula 4 or a compound represented by structural formula 4′.

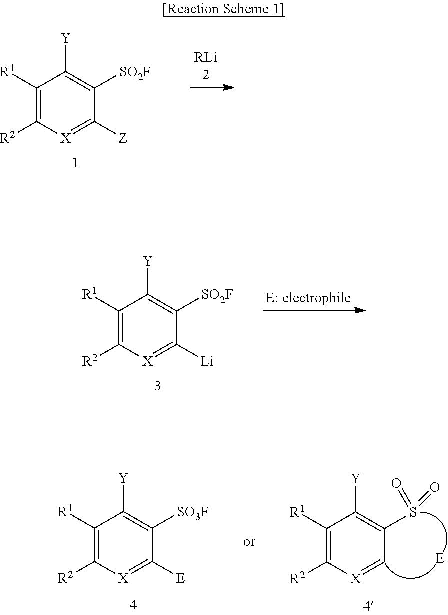

in the reaction scheme 1, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group, X is a carbon atom or a nitrogen atom, Y is a hydrogen atom, a fluorine atom, a chlorine atom, a bromine atom, or an iodine atom, Z is a fluorine atom, a chlorine atom, a bromine atom, or an iodine atom, R is C1 to C9 alkyl group of or C6 to C14 aryl group, and E is an electrophile.

In addition, the method may be an ultra-fast synthesizing method using ultra-fast flow of the reactants.

In addition, the electrophile may be substituted with lithium at the location of ortho position of the sulfonyl fluoride group of the intermediate, or the electrophile is substituted with lithium at the location of ortho position of the sulfonyl fluoride of the intermediate and is substituted with fluorine atom of the sulfonyl fluoride, thus an intra-molecular cyclization reaction being carried out.

In addition, the method may be carried out in a flow-based capillary microreactor.

In addition, the step (a) may be carried out for 0.016 to 6.3 seconds, and the step (b) is carried out for 1.5 to 3 seconds.

In addition, the electrophile of the step (b) may comprise at least one selected from the group consisting of t-BuOH, methyl triflate, chlorotrimethylsilane, tributyltin chloride, methyl chloroformate, iodine, N-fluorobenzenesulfonimide, isopropoxy-4,4,5,5-tetramethyl-1,3,2-dioxaborolane, benzaldehyde, 4-formylbenzonitrile, phenyl isocyanate, and methyl isocyanate.

In addition, the product of step (b) may comprise at least one selected from the group consisting of

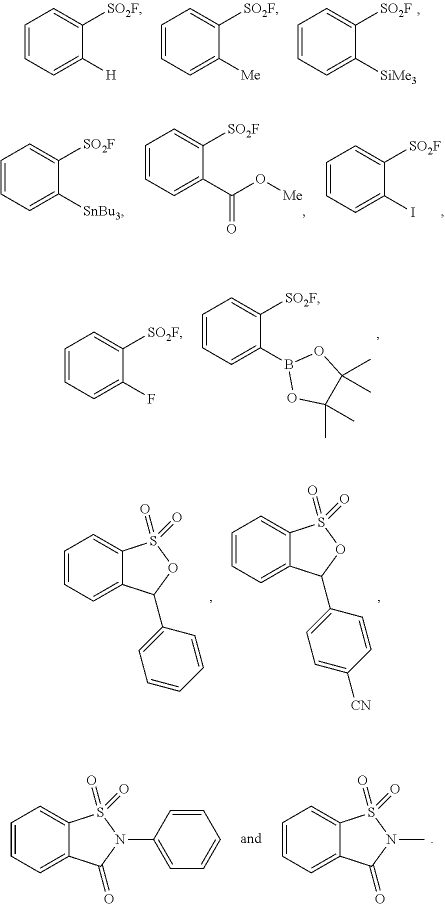

In addition, the steps (a) and (b) may be each independently carried out at a temperature in a range of −58 to 25° C.

In addition, the method may further comprise: (c) in reaction scheme 2, the compound represented by structural formula 4 is reacted with a compound represented by structural formula 5 to synthesize a compound represented by structural formula 6, wherein the step (c) is carried out after the step (b).

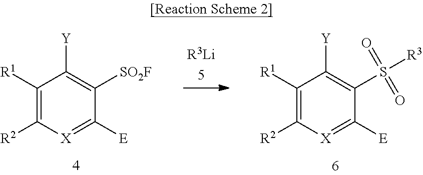

in the reaction scheme 2, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group, X is a carbon atom or a nitrogen atom, Y is a hydrogen atom, a fluorine atom, a chlorine atom, a bromine atom, or an iodine atom, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and E is an electrophile.

In addition, the compound represented by the structural formula 5 may comprise at least one selected from the group consisting of

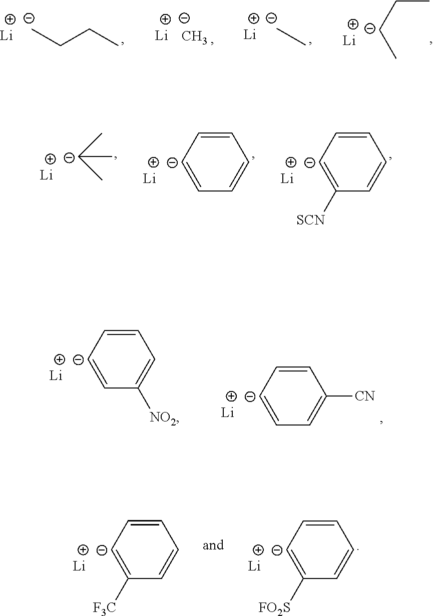

In addition, the compounds represented by the structural formula 6 may comprise at least one selected from the group consisting of

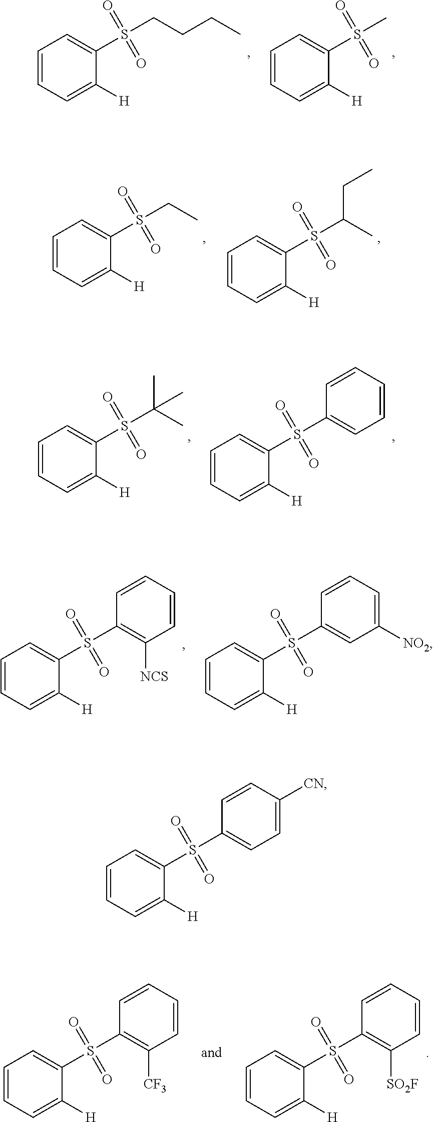

In addition, the method further may comprise: (a′) reacting a compound represented by structural formula 5a with a compound represented by structural formula 5b, thus synthesizing a compound represented by structural formula 5 in reaction scheme 3, wherein the step (a′) is carried out before the step (c).

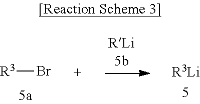

in the reaction scheme 3, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and R′ is C1 to C9 alkyl group or C6 to C14 aryl group.

FIG. 7A shows a procedure for the Br—Li exchange reaction of 2-bromobenzenesulfonyl fluoride (1a) followed by reaction with tert-butanol and subsequent SuFEx reaction with a commercially available organolithium nucleophile in a capillary microreactor, and FIG. 7B shows a procedure for the Br—Li exchange reaction of 2-bromobenzenesulfonyl fluoride (1a) followed by reaction with an electrophile and subsequent SuFEx reaction with an organolithium nucleophile generated in-situ in a capillary microreactor.

Referring to FIGS. 7A and 7B, there is provided an ultra-fast synthesis apparatus of an ortho-functionalized benzenesulfonyl fluoride derivative, the apparatus may comprise a first micromixer M1 which prepares a first mixture by mixing a benzenesulfonyl fluoride derivative represented by Formula 1 and an organolithium represented by structural formula 2 in reaction scheme 1; a first microreactor R1 which receives the first mixture from the first micromixer M1 and reacts it to synthesize an intermediate represented by structural formula 3; a second micromixer M2 which prepare a second mixture by mixing the intermediate supplied from the first microreactor R1 and an electrophile; and a second microreactor R2 which receives the second mixture from the second micromixer M2 and reacts it to synthesize a compound represented by structural formula 4 or a compound represented by structural formula 4′.

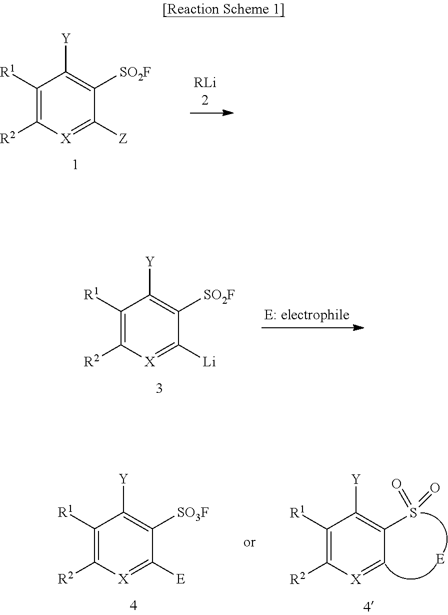

in the reaction scheme 1, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group, X is a carbon atom or a nitrogen atom, Y is a hydrogen atom, a fluorine atom, a chlorine atom, a bromine atom, or an iodine atom, Z is a fluorine atom, a chlorine atom, a bromine atom, or an iodine atom, R is C1 to C9 alkyl group of or C6 to C14 aryl group, and E is an electrophile.

In addition, the reaction in the first microreactor R1 may be carried out for a duration of 0.016 to 6.3 seconds.

In addition, the synthesis reaction in the second microreactor R2 may be carried out for a duration of 1.5 to 3 seconds.

In addition, the reaction in the first microreactor R1 and in the second microreactor R2 may be carried out at a temperature in a range of −58 to 25° C. respectively.

In addition, the ultra-fast synthesis apparatus may further comprise a third micromixer M3 which prepares a third mixture by mixing a compound represented by structural formula 4 and a compound represented by structural formula 5 in reaction scheme 2; and a third microreactor R3 which reacts the third mixture supplied from the third micromixer M3 to synthesize a compound represented by structural formula 6.

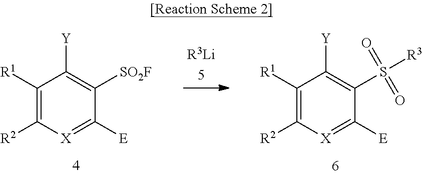

in the reaction scheme 2, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group, X is a carbon atom or a nitrogen atom, Y is a hydrogen atom, a fluorine atom, a chlorine atom, a bromine atom, or an iodine atom, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and E is an electrophile.

In addition, the reaction in the third microreactor R3 may be carried out for 5 to 10 seconds.

In addition, ultra-fast synthesis apparatus may further comprise a fourth micromixer M4 which prepares a fourth mixture by mixing a compound represented by structural formula 5a and a compound represented by structural formula 5b in reaction scheme 3; and a fourth microreactor R4 that reacts the fourth mixture supplied from the fourth micromixer M4 to synthesize a compound represented by structural formula 5.

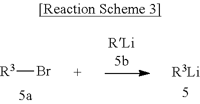

in the reaction scheme 3, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and R′ is C1 to C9 alkyl group or C6 to C14 aryl group.

In addition, the reaction in the fourth microreactor R4 may be carried out for 0.016 to 6.3 seconds.

The present disclosure enables the synthesis of several functionalized sulfonyl fluorides in a few seconds under mild temperature conditions in addition to the next-generation click chemistry Sulfonyl Fluoride Exchange (SuFEx) chemistry, thereby efficiently securing sulfonyl fluoride, the main reactant of the SuFEx reaction.

In addition, effective reactant synthesis can be facilitated in the fields of drug discovery, biosensors, bioorthogonal chemistry, or drug delivery through combinatorial chemistry through the present disclosure.

In addition, a new, fast and efficient synthesis route for the synthesis of the benzooxathiol oxide and saccharin structure, including the precursor of the neuroprotective drug lepinotan, can be made available through the present disclosure.

### BRIEF DESCRIPTION OF THE DRAWINGS

Since the accompanying drawings are for reference in describing exemplary Examples of the present disclosure, the technical spirit of the present should not be construed as being limited to the accompanying drawings, in which:

FIG. 1A shows a schematic diagram of the synthesis method of the ortho-functionalized benzenesulfonyl fluoride derivative in a capillary microreactor and the effect of temperature and residence time of R1 (t^{R1}) and R2 (t^{R2}).

FIG. 1B shows an effect of temperature and residence time of R1 (t^{R1}) on the yield of the protonated product (2aa) in the Br—Li exchange reaction of 2-bromobenzene sulfonyl fluoride (1a) in a capillary microreactor was revealed, FIG. 1C shows the effect of temperature and residence time of R1(t^{R1}) on the yield of the protonated product (2da) in the Br—Li exchange reaction of 2-bromo-3-pyridinesulfonyl fluoride (1d) in a capillary microreactor, and FIG. 1D show the effect of temperature and residence time of R1 (t^{R1}) on the yield of the protonated product 2ea in the Br—Li exchange reaction of 2-bromo-6-fluorobenzensulfonyl fluoride (1e) in a capillary microreactor, wherein the numbers in the contour map are the yields determined by ¹H NMR spectroscopy using 1,3,5-trimethoxybenzene as the internal standard.

FIG. 2 shows a synthesis of functionalized sulfonyl fluoride through the preparation of aryl lithium intermediates under optimal conditions in a capillary microreactor;

FIG. 3 shows a control of aryllithium intermediates in a capillary microreactor demonstrated the synthesis of lepinotan precursors as a neuroprotective drug to reduce brain damage after extracephalic drugs;

FIG. 4 shows a one-flow integrated synthesis including ultra-fast functionalization of aryl sulfonyl fluoride and SuFEx reaction with various organolithium nucleophiles;

FIG. 5 shows a single-flow integrated synthesis of sulfones (4) containing tributyltin and sulfonyl fluoride groups for further chemical transformation;

FIG. 6 shows an effect of increasing n-BuLi equivalents and decreasing t-BuOH equivalents on the yields of benzenesulfonyl fluoride (2a) and SuFEx product (3a).

FIG. 7A shows procedure for Br—Li exchange reaction of 2-bromobenzenesulfonyl fluoride (1a) followed by reaction with tert-Butanol and subsequent SuFEx reaction with commercially available organolithium nucleophile in a capillary microreactor.

FIG. 7B shows procedure for Br—Li exchange reaction of 2-bromobenzenesulfonyl fluoride (1a) followed by reaction with electrophiles and subsequent SuFEx reaction with in situ generated organolithium nucleophile in a capillary microreactor.

FIG. 8 shows an optical image of a capillary microreactor consisting of micromixers (M1-M4) and microtube reactors (R1-R4) for aryllithium reaction and SuFEx coupling using on-site generated organolithium nucleophiles are shown.

### DESCRIPTION OF THE PREFERRED EXAMPLES

Herein after, examples of the present disclosure will be described in detail with reference to the accompanying drawings in such a manner that the ordinarily skilled in the art can easily implement the present disclosure.

The description given below is not intended to limit the present disclosure to specific Examples. In relation to describing the present disclosure, when the detailed description of the relevant known technology is determined to unnecessarily obscure the gist of the present disclosure, the detailed description may be omitted.

The terminology used herein is for the purpose of describing particular examples only and is not intended to limit the scope of the present disclosure. As used herein, the singular forms “a”, “an”, and “the” are intended to comprise the plural forms as well unless the context clearly indicates otherwise. It will be further understood that the terms “comprise” or “have” when used in the present disclosure specify the presence of stated features, integers, steps, operations, elements and/or combinations thereof, but do not preclude the presence or addition of one or more other features, integers, steps, operations, elements, and/or combinations thereof.

Terms including ordinal numbers used in the specification, “first”, “second”, etc. can be used to discriminate one component from another component, but the order or priority of the components is not limited by the terms unless specifically stated. These terms are used only for the purpose of distinguishing a component from another component. For example, without departing from the scope of the present disclosure, a first component may be referred as a second component, and a second component may be also referred to as a first component.

In addition, when it is mentioned that a component is “formed” or “stacked” on another component, it should be understood such that one component may be directly attached to or directly stacked on the front surface or one surface of the other component, or an additional component may be disposed between them.

As used herein, unless otherwise defined, the term “valence bond” means a single bond, a double bond or a triple bond.

As used herein, unless otherwise defined, the term “substituted” means that at least one hydrogen on a substituent or a compound is substituted with deuterium, a halogen group, a hydroxyl group, an amino group, a C1 to C30 amine group, a nitro group, a silyl group, a C1 to C30 alkyl group, a C1 to C30 alkylsilyl group, a C3 to C30 cycloalkyl group, a C1 to C30 heterocycloalkyl group, a C6 to C30 aryl group, a C1 to C30 heteroaryl group, a C1 to C20 alkoxy group, a C1 to C10 trifluoroalkyl group, a thiocyanate group, a sulfonyl fluoride group, or a cyano group.

Further, among the halogen group, the hydroxyl group, the amino group, the C1 to C30 amine group, the silyl group, the C1 to C30 alkyl group, the C1 to C30 alkylsilyl group, the C3 to C30 cycloalkyl group, the C6 to C30 aryl group, the C1 to C20 alkoxy group, the C1 to C10 trifluoroalkyl group, the thiocyanate group, the sulfonyl fluoride group, or the cyano group, which is substituted, two adjacent substituents may be fused to form a ring.

As used herein, unless otherwise defined, the term “hetero” means a functional group containing 1˜4 heteroatoms selected from the group consisting of N, O, S and P, the remainder being carbon.

As used herein, unless otherwise defined, the term “combination thereof” means that two or more substituents are coupled with each other by a linker or two or more substituents are condensed to each other.

As used herein, unless otherwise defined, the term “hydrogen” means hydrogen, deuterium or tritium.

As used herein, unless otherwise defined, the term “alkyl group” means an aliphatic hydrocarbon group.

The alkyl group may be a “saturated alkyl group” without any double bond or triple bond.

The alkyl group may be an “unsaturated alkyl group” with at least one double bond or triple bond.

The term “alkenylene group” means a functional group having at least one carbon-carbon double bond between at least two carbon atoms, and the term “alkynylene group” means a functional group having at least one carbon-carbon triple bond between at least two carbon atoms. The alkyl group may be branched, linear or cyclic, regardless of whether it is saturated or unsaturated.

The alkyl group may be a C1 to C30 alkyl group, preferably a C1 to C20 alkyl, more preferably a C1 to C10 alkyl group, and much more preferably a C1 to C6 alkyl group.

For example, a C1 to C4 alkyl group indicates an alkyl chain containing 1˜4 carbon atoms, particularly an alkyl chain which is selected from the group consisting of methyl, ethyl, propyl, iso-propyl, n-butyl, iso-butyl, sec-butyl and t-butyl.

Specific examples of the alkyl group include a methyl group, an ethyl group, a propyl group, an isopropyl group, a butyl group, an isobutyl group, a t-butyl group, a pentyl group, a hexyl group, an ethenyl group, a propenyl group, a butenyl group, a cyclopropyl group, a cyclobutyl group, a cyclopentyl group, a cyclohexyl group, etc.

The “amine group” includes an arylamine group, an alkylamine group, an arylalkylamine group, or an alkylarylamine group.

The term “cycloalkyl group” refers to a monocyclic or fused-ring polycyclic (i.e., rings which share adjacent pairs of carbon atoms) functional group.

The term “heterocycloalkyl group” means a cycloalkyl group containing 1˜4 heteroatoms selected from the group consisting of N, O, S and P, the remainder being carbon. In the case where the heterocycloalkyl group is a fused ring, at least one ring may contain 1˜4 heteroatoms.

The term “aromatic group” means a cyclic functional group where all ring atoms have p-orbitals, and these p-orbitals form conjugation. Specific examples thereof include an aryl group and a heteroaryl group.

The term “aryl group” refers to a monocyclic or fused-ring polycyclic (i.e., rings which share adjacent pairs of carbon atoms) functional group.

The term “heteroaryl group” means an aryl group containing 1˜4 heteroatoms selected from the group consisting of N, O, S and P, the remainder being carbon. In the case where the heteroalkyl group is a fused ring, at least one ring may contain 1˜4 heteroatoms.

In the aryl group and the heteroaryl group, the number of ring atoms is the sum of the number of carbons and the number of non-carbon atoms.

When alkyl and aryl are used in combination as in “alkylaryl group” or “arylalkyl group,” “alkyl” and “aryl” respectively have the meanings as above.

The term “arylalkyl group” means an aryl substituted alkyl radical such as benzyl, and is incorporated in the alkyl group.

The term “alkylaryl group” means an alkyl substituted aryl radical, and is incorporated in the aryl group.

Hereinafter, the embodiment of the present disclosure shall be explained with reference to the attached drawing, and in describing it by reference to the accompanying drawing, the same or corresponding components shall be given the same figure number and the duplicate description thereof shall be omitted.

The ultra-fast flow synthesis of functionalized sulfonyl fluorides and subsequent sufex connections via lithiated chemistry will be described in detail. However, those are described as examples, and the present disclosure is not limited thereto and is only defined by the scope of the appended claims. In addition, in the present disclosure, the respective yield is determined by ¹H NMR spectroscopy using 1,3,5-trimethoxybenzene as an internal standard.

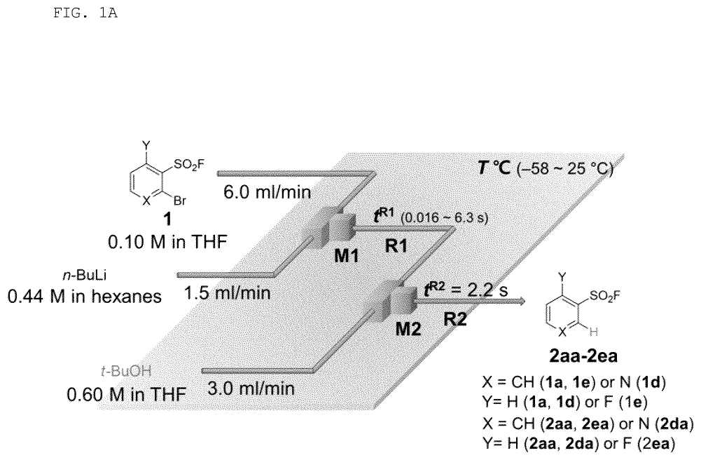

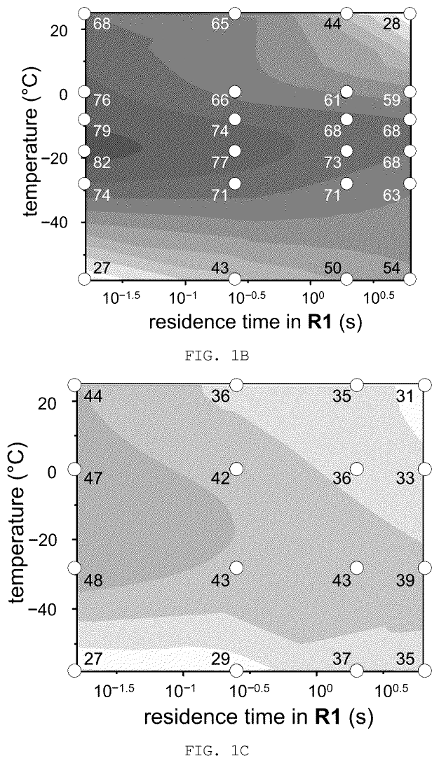

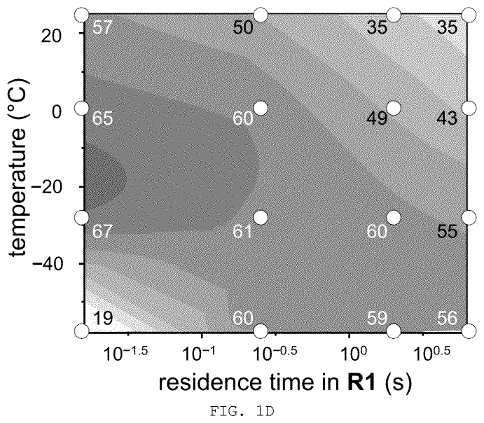

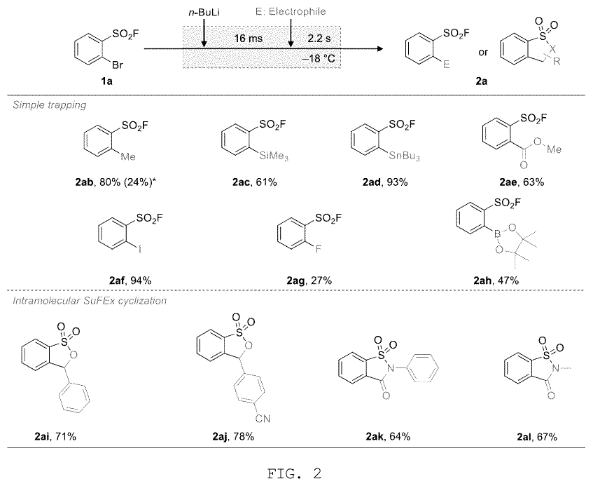

FIG. 1A shows a schematic diagram of the synthesis method of the ortho-functionalized benzenesulfonyl fluoride derivative in a capillary microreactor and the effect of temperature and residence time of R1 (t^{R1}) and R2 (t^{R2}); FIG. 2 is a synthesis of functionalized sulfonyl fluoride through the preparation of aryl lithium intermediates under optimal conditions in a capillary microreactor.

Referring to FIGS. 1A and 2, the present disclosure is provided a method of synthesizing an ortho-functionalized benzenesulfonyl fluoride derivative, the method comprising: (a) reacting a benzenesulfonyl fluoride derivative represented by structural formula 1 with an organolithium represented by structural formula 2, thus synthesizing an intermediate represented by structural formula 3 in reaction scheme 1; and (b) reacting the intermediate with an electrophile, thus synthesizing a compound represented by structural formula 4 or a compound represented by structural formula 4′.

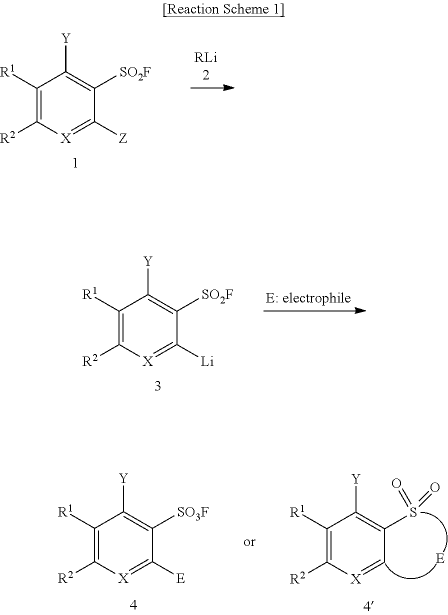

in the reaction scheme 1, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group, X is a carbon atom or a nitrogen atom, Y is a hydrogen atom, a fluorine atom, a chlorine atom, a bromine atom, or an iodine atom, Z is a fluorine atom, a chlorine atom, a bromine atom, or an iodine atom, R is C1 to C9 alkyl group of or C6 to C14 aryl group, and E is an electrophile.

In addition, the method may be an ultra-fast synthesizing method using ultra-fast flow of the reactants.

In addition, the electrophile may be substituted with lithium at the location of ortho position of the sulfonyl fluoride group of the intermediate, or the electrophile is substituted with lithium at the location of ortho position of the sulfonyl fluoride of the intermediate and is substituted with fluorine atom of the sulfonyl fluoride, thus an intra-molecular cyclization reaction being carried out.

In addition, the method may be carried out in a flow-based capillary microreactor.

In addition, the step (a) may be carried out for a duration of 0.016 to 6.3 seconds, and preferably for a duration of 0.016 seconds, and step (b) may be carried out for 1.5 to 3 seconds, and preferably 2.2 seconds. When the duration of step (a), i.e., the residence time of the aryllithium intermediate, is 0.016 seconds, the aryllithium intermediate having the sulfonyl fluoride functional group can be controlled most efficiently. The residence time can be utilized to synthesize several activated sulfonyl fluorides using various electrophiles. When the duration of step (b), i.e., the time during which sulfonyl fluoride is synthesized, is shorter than 1.5 seconds, the aryllithium intermediate does not react sufficiently with the lipophilic body and most of it is degraded, which is undesirable. When the duration is longer than 3 seconds, the reaction time is unnecessarily long, which is undesirable.

In addition, the electrophile of the step (b) may comprise at least one selected from the group consisting of t-BuOH, methyl triflate, chlorotrimethylsilane, tributyltin chloride, methyl chloroformate, iodine, N-fluorobenzenesulfonimide, isopropoxy-4,4,5,5-tetramethyl-1,3,2-dioxaborolane, benzaldehyde, 4-formylbenzonitrile, phenyl isocyanate, and methyl isocyanate. The use of aldehyde or isocyanate-based electrophile allows for the synthesis of benzooxathiol dioxide and saccharin structures in a way that is not possible with batch reactors such as flasks.

In addition, the product of step (b) may comprise at least one selected from the group consisting of

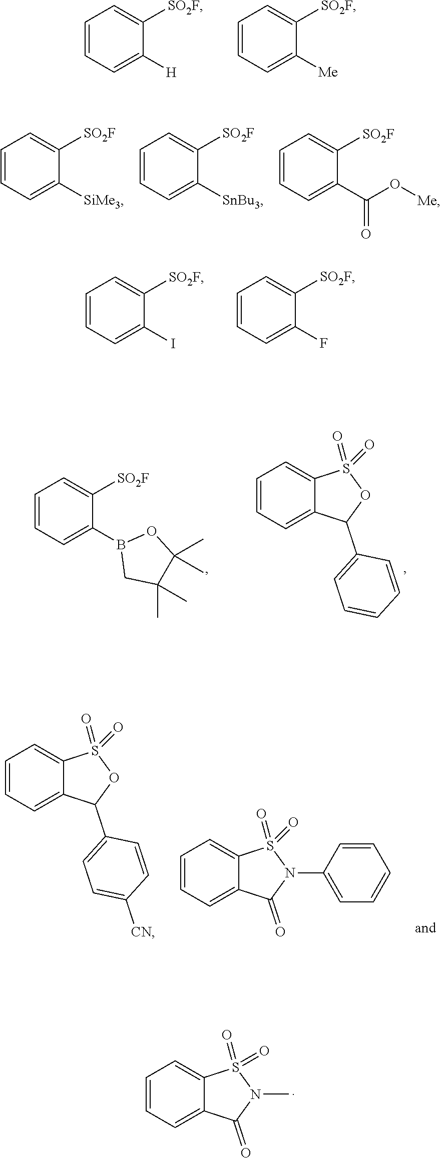

In addition, the steps (a) may be carried out at a temperature in a range of −58 to 25° C. When the temperature of step (a) is below −58° C., the generation of the aryllithium intermediate does not proceed smoothly, which is undesirable. When the temperature is higher than 25° C., the decomposition of the aryllithium intermediate is accelerated and it becomes difficult to control, which is undesirable.

In addition, the steps (b) may be carried out at a temperature in a range of −58 to 25° C., preferably −20 to −15° C., and more preferably −18° C. When the temperature is lower than −58° C., the generation of aryl lithium intermediate is relatively small, but it does not proceed smoothly, which is undesirable. When the temperature is higher than 25° C., the decomposition of the aryllithium intermediate is relatively small, but it is accelerated and it is difficult to control, which is undesirable.

In addition, when the isocyanate compound is methyl isocyanate, a precursor of repinotan may be synthesized.

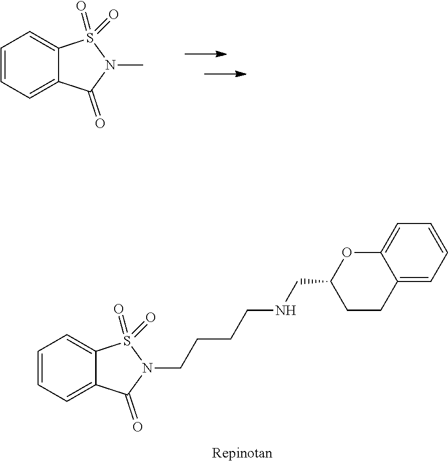 [SMILES: CN1C(=O)c2ccccc2S1(=O)=O ; O=C1c2ccccc2S(=O)(=O)N1CCCCNC[C@H]1CCc2ccccc2O1]

In addition, the precursor of the lepinotan can be obtained with a yield of 60 to 70% within 1.5 to 3 seconds. The present disclosure is capable of synthesizing a precursor of lepinotan in less than 3 seconds under milder temperature conditions (−18° C.) than conventional techniques.

In addition, the present disclosure may control the selectivity of the product by controlling the concentration.

In addition, the method may further comprise: (c) in reaction scheme 2, the compound represented by structural formula 4 is reacted with a compound represented by structural formula 5 to synthesize a compound represented by structural formula 6, wherein the step (c) is carried out after the step (b).

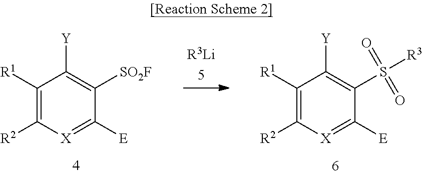

in the reaction scheme 2, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group, X is a carbon atom or a nitrogen atom, Y is a hydrogen atom, a fluorine atom, a chlorine atom, a bromine atom, or an iodine atom, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and E is an electrophile.

In addition, the compound represented by the structural formula 5 may comprise at least one selected from the group consisting of

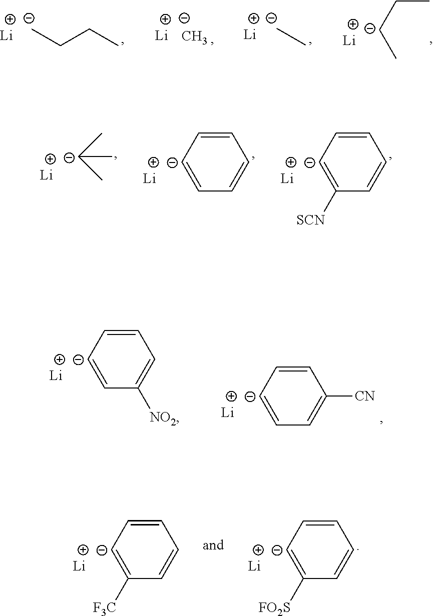

In addition, the compounds represented by the structural formula 6 may comprise at least one selected from the group Consisting of

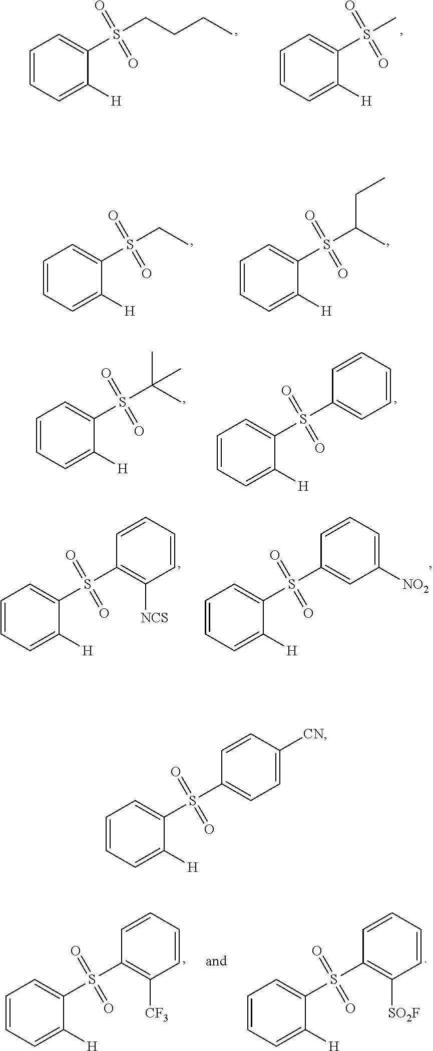

In addition, the method further may comprise: (a′) reacting a compound represented by structural formula 5a with a compound represented by structural formula 5b, thus synthesizing a compound represented by structural formula 5 in reaction scheme 3, wherein the step (a′) is carried out before the step (c).

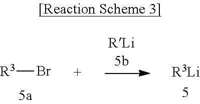

in the reaction scheme 3, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and R′ is C1 to C9 alkyl group or C6 to C14 aryl group.

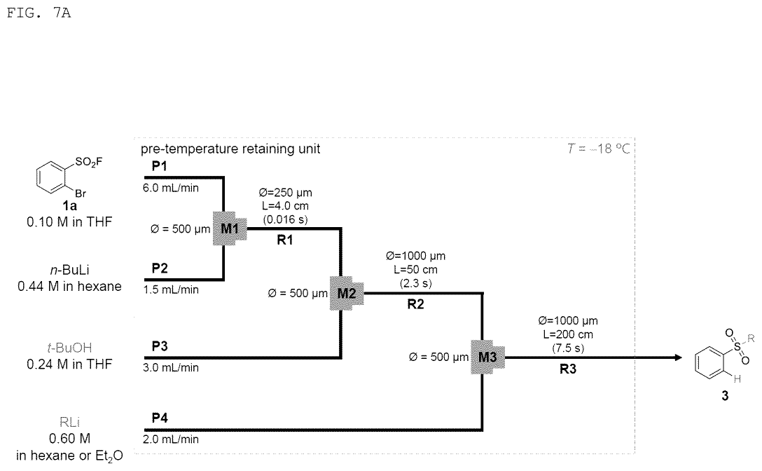

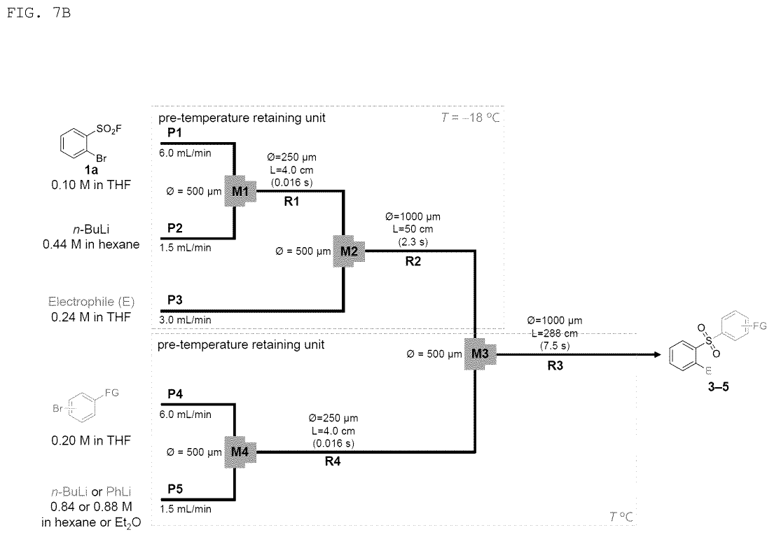

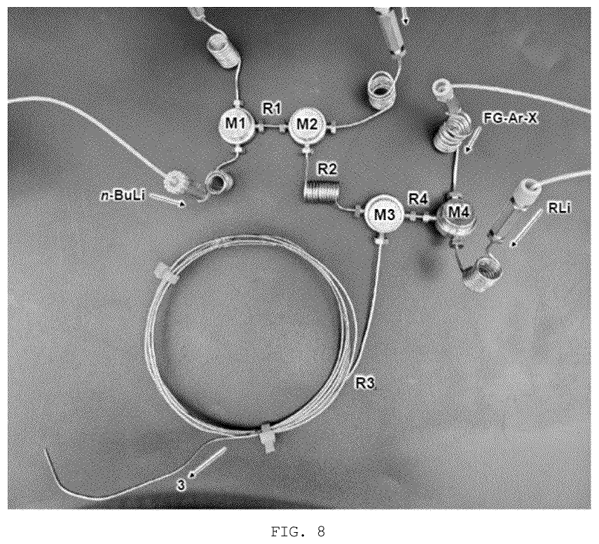

FIG. 7A shows a procedure for the Br—Li exchange reaction of 2-bromobenzenesulfonyl fluoride (1a) followed by reaction with tert-butanol and subsequent SuFEx reaction with a commercially available organolithium nucleophile in a capillary microreactor, FIG. 7B shows a procedure for the Br—Li exchange reaction of 2-bromobenzenesulfonyl fluoride (1a) followed by reaction with an electrophile and subsequent SuFEx reaction with an organolithium nucleophile generated in-situ in a capillary microreactor, and FIG. 8 shows an optical images of a capillary microreactor consisting of micromixers (M1-M4) and microtube reactors (R1-R4) for aryllithium reaction and SuFEx coupling using on-site generated organolithium nucleophiles are shown.

Referring to FIGS. 7A, 7B and 8, there is provided an ultra-fast synthesis apparatus of an ortho-functionalized benzenesulfonyl fluoride derivative, the apparatus comprises a first micromixer M1 which prepares a first mixture by mixing a benzenesulfonyl fluoride derivative represented by Formula 1 and an organolithium represented by structural formula 2 in reaction scheme 1; a first microreactor R1 which receives the first mixture from the first micromixer M1 and reacts it to synthesize an intermediate represented by structural formula 3; a second micromixer M2 which prepare a second mixture by mixing the intermediate supplied from the first microreactor R1 and an electrophile; and a second microreactor R2 which receives the second mixture from the second micromixer M2 and reacts it to synthesize a compound represented by structural formula 4 or a compound represented by structural formula 4′.

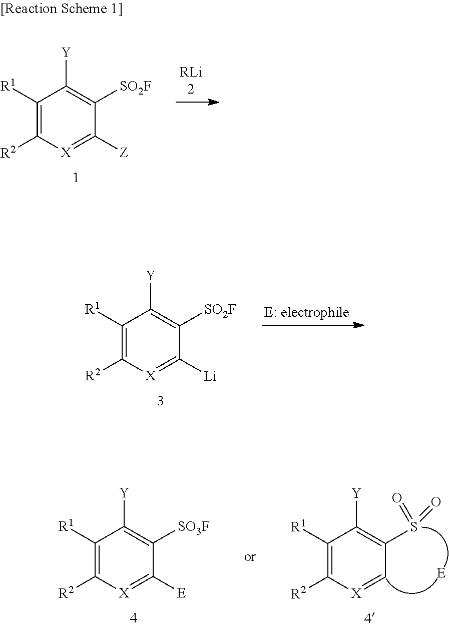

in the reaction scheme 1, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group, X is a carbon atom or a nitrogen atom, Y is a hydrogen atom, a fluorine atom, a chlorine atom, a bromine atom, or an iodine atom, Z is a fluorine atom, a chlorine atom, a bromine atom, or an iodine atom, R is C1 to C9 alkyl group of or C6 to C14 aryl group, and E is an electrophile.

In addition, the reaction in the first microreactor R1 may be carried out for a duration of 0.016 to 6.3 seconds. When the duration is shorter than 0.016 seconds, the Br—Li exchange reaction does not occur sufficiently, which is undesirable. When the duration is longer than 6.3 seconds, the time between the formation of the aryllithium intermediate and the meeting with the lipophilic body is prolonged, and it is decomposed, which is undesirable.

In addition, the synthesis reaction in the second microreactor R2 may be carried out for a duration of 1.5 to 3 seconds. When the duration is shorter than 1.5 seconds, it is difficult for the aryllithium intermediate to react sufficiently with the electrophilic body, which is undesirable. When the duration is longer than 3 seconds, the reaction time is unnecessarily long, which is undesirable.

In addition, the reactions in the first microreactor R1 and in the second microreactor R2 may be carried out at a temperature in a range of −58 to 25° C. respectively. When the temperature is lower than −58° C., the generation of the aryllithium intermediate does not proceed smoothly, which is undesirable. When the temperature is higher than 25° C., the decomposition of the aryllithium intermediate is accelerated and it becomes difficult to control, which is undesirable.

In addition, the ultra-fast synthesis apparatus may further comprise a third micromixer M3 which prepares a third mixture by mixing a compound represented by structural formula 4 and a compound represented by structural formula 5 in reaction scheme 2; and a third microreactor R3 which reacts the third mixture supplied from the third micromixer M3 to synthesize a compound represented by structural formula 6.

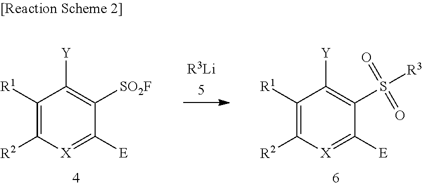

in the reaction scheme 2, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group, X is a carbon atom or a nitrogen atom, Y is a hydrogen atom, a fluorine atom, a chlorine atom, a bromine atom, or an iodine atom, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and E is an electrophile.

In addition, the reaction in the third microreactor R3 may be carried out for a duration of 5 to 10 seconds. When the duration is shorter than 5 seconds, the generation of the aryllithium intermediate and the electrophilic body, and the reaction with the organolithium reagent are not sufficiently advanced, which is undesirable. When the duration is longer than 10 seconds, the time between the generation of the aryllithium intermediate and the encounter with the electrophile is long, and it will decompose or the reaction time will be unnecessarily long, which is undesirable.

In addition, the ultra-fast synthesis apparatus may further comprise a fourth micromixer M4 which prepares a fourth mixture by mixing a compound represented by structural formula 5a and a compound represented by structural formula 5b in reaction scheme 3; and a fourth microreactor R4 that reacts the fourth mixture supplied from the fourth micromixer M4 to synthesize a compound represented by structural formula 5.

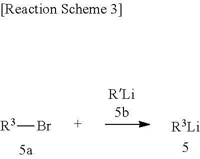

in the reaction scheme 3, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and R′ is C1 to C9 alkyl group or C6 to C14 aryl group.

In addition, the reaction in the fourth microreactor R4 may be carried out for a duration of 0.016 to 6.3 seconds, and preferably 0.016 seconds. When the reaction in the fourth microreactor R4 is 0.016 seconds, the aryllithium intermediate having a sulfonyl-fluoride functional group can be most efficiently controlled. The residence time can be utilized to synthesize several functionalized sulfonyl fluorides using various electrophiles.

In addition, the functionalized aryl sulfonyl derivative can be combined into a one-flow. By introducing a variety of ultra-short-lived aryllithium intermediates that need to be controlled by commercially available organolithium reagent or microreactor in the one-flow, the targeted functionalized aryl sulfonyl derivative can be synthesized with high selectivity in less than 10 seconds.

In addition, when the functionalized aryl sulfonyl derivative is synthesized, it may contain an aryllithium intermediate.

In addition, the electrophile is tributyltin chloride, and may comprise a functionalized aryl sulfonyl derivative represented by structural formula below synthesized by the introduction of an aryllithium intermediate having a sulfonyl fluoride functional group.

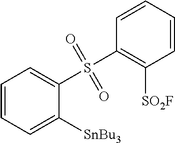

In addition, the productivity of the functionalized aryl sulfonyl derivative represented by the structural formula above may be 10 to 20 g/h, and preferably 14 to 15 g/h. The functionalized aryl sulfonyl derivative synthesized within 10 seconds has a productivity of 14.4 g per hour. Since the tributyltin and sulfonyl fluoride functional groups are capable of further chemical modification through coupling reaction and another SuFEx reaction, respectively, the present invention can synthesize very useful compounds.

The present invention may enable the efficient securing of sulfonyl fluoride, a main reactant of the SuFEx reaction, by enabling the synthesis of various functionalized sulfonyl fluorides under mild temperature conditions within seconds in addition to the next-generation click chemistry, sulfonyl fluoride exchange (SuFEx) chemistry. Based on this, it is possible to easily synthesize effective reactants in the fields of new drug development, biosensors, and bioorthogonal chemistry or drug delivery systems through combinatorial chemistry. In addition, it may enable a new, fast and efficient synthetic route for the synthesis of benzoxathiol dioxide and saccharin structures, including precursors of the neuroprotective drug lepinotan.

### EXAMPLE

Hereinafter, the examples of the present invention will be described. However, the examples are for illustrative purposes, and the scope of the present invention is not limited by the examples.

Tetrahydrofuran (THF), n-hexane, and diethyl ether (Et₂O) were purchased from Sigma-Aldrich as a dry solvent and used without further purification. 2-Bromobenzenesulfonyl chloride, 3-bromobenzenesulfonyl chloride, 4-bromobenzenesulfonyl chloride, iodine, N-fluorobenzenesulfonimide, 4-formylbenzonitrile, 1-iodo-3-nitrobenzene, 4-bromobenzonitrile, and 2-bromobenzotrifluoride were purchased from Tokyo Chemical Industry Co. Ltd. Potassium hydrogenfluoride, methanol, tert-butanol, iodomethane, methyl trifluoromethanesulfonate, chlorotrimethylsilane, tributyltin chloride, methyl chloroformate, 2-isopropoxy-4,4,5,5-tetramethyl-1,3,2-dioxaborolane, benzaldehyde, phenyl isocyanate, methyllithium (MeLi, 1.6 M in diethyl ether), ethyllithium (EtLi, 0.5 M in benzene: cyclohexane), n-butyllithium (n-BuLi, 2.5 M in hexanes), sec-butyllithium (s-BuLi, 1.4 M in cyclohexane), tert-butyllithium (t-BuLi, 1.7 M in pentane), phenyllithium (PhLi, 1.9 M in dibutyl ether), and 2-bromophenyl isothiocyanate were purchased from Sigma-Aldrich. Unless otherwise noted, all commercial materials were used without further purification.

### Capillary Microreactor

Stainless steel (SUS316) microtube reactors with inner diameters of 250 and 1000 μm were purchased from GL Science and cut into appropriate lengths (inner diameter 0=250 μm, length L=4 cm; inner diameter Ø=1000 μm, length L=4, 30, 50, 100, 200 and 288 cm). Stainless steel (SUS304) T-shaped micromixers with inner diameter of 500 μm were purchased from VICI JOUR Co. The micromixers and the microtube reactors were connected with stainless steel fittings (GL Science, 1/16″ OUW) to construct the microreactor system.

FIG. 7A shows a procedure for the Br—Li exchange reaction of 2-bromobenzenesulfonyl fluoride (1a) followed by reaction with tert-butanol and subsequent SuFEx reaction with a commercially available organolithium nucleophile in a capillary microreactor, and FIG. 7B shows a procedure for the Br—Li exchange reaction of 2-bromobenzenesulfonyl fluoride (1a) followed by reaction with an electrophile and subsequent SuFEx reaction with an organolithium nucleophile generated in-situ in a capillary microreactor.

Referring to FIGS. 7A and 7B, a capillary microreactor consisting of four T-shaped micromixers (M1, M2, M3 and M4), four microtube reactors (R1, R2, R3 and R4), and five tube pre-temperature retaining units (P1, P2, P3, P4 and P5 (inner diameter Ø=1000 μm, length L=50 cm)) was used. A 0.10 M solution of a 2-benzenesulfonyl fluoride (1a) in THF (flow rate: 6.0 mL min⁻¹) and a 0.44 M solution of n-BuLi in hexane (flow rate: 1.5 mL min⁻¹) were introduced to M1 (Ø=500 μm) by syringe pumps. The resulting solution was passed through R1 and was mixed with a 0.24 M solution of electrophile in THF (flow rate: 3.0 mL min⁻¹) in M2 (Ø=500 μm). The resulting solution was passed through R2 (Ø=1000 μm, L=50 cm). A 0.20 M solution of a functionalized bromobenzene in THF (flow rate: 6.0 mL min⁻¹) and a 0.84 M (or 0.88 M for 1a) solution of n-BuLi (or PhLi for 1-iodo-3-nitrobenzne) in hexane (or Et₂O for PhLi) (flow rate: 1.5 mL min⁻¹) were introduced to M3 (Ø=500 μm) by syringe pumps. The resulting solution was passed through R3 and was mixed with the resulting solution comes from R2 in M4 (Ø=500 μm). The resulting solution was passed through R4 (Ø=1000 μm, L=288 cm). After a steady state was reached, the product solution was collected for 30 s while being quenched with sat. NH₄C1 aqueous solution. The reaction mixture was analyzed by GC. The organic phase was separated and the aqueous phase was extracted with ethyl acetate. After the combined organic phase was dried over Na₂SO₄, solvent was removed. The product was analyzed by ¹H, ¹³C and ¹⁹F NMR and GCMS.

The capillary microreactors were placed in cooling bath to control the temperature. The reagents were continuously injected to the capillary microreactor using PHD Ultra syringe pump Harvard Apparatus, equipped with gas tight syringes (50 mL, inner diameter: 27.6 mm) purchased from SGE Analytical Science. After a steady state was reached, the product solution was collected for 30 s unless otherwise noted.

Based on the above results, the inventors performed Br—Li exchange and trapped the intermediate with an electrophile using a capillary microreactor to precisely control the intermediate (FIG. 1A). In our endeavor to enhance subsequent reactions of the generated intermediate with electrophiles, the inventors meticulously screened residence times and temperatures to investigate their effects on the yield, as determined by ¹H NMR spectroscopy. The o-lithiated benzenesulfonyl fluoride intermediate exhibited increased decomposition with prolonged residence time in R1, except when screened at −58° C. Conversely, under the −58° C. condition, yields were increased with elongated residence time in R1, probably because increased Br—Li exchange progress outweighed the intermediate decomposition. Multiple screenings of the entire conditions led to the identification of optimal conditions (t^{R1}=0.016 s, T=−18° C.) that provided the highest yield of 82% (FIG. 1B). It is noteworthy that the presented synthetic methodology afforded the desired sulfonyl fluoride within seconds under mild temperatures as well as in dramatically improved yields. Additionally, the inventors attempted reaction condition screenings for m- and p-lithiated intermediates using the capillary microreactor, but unfortunately, only a trace amount of the desired product was obtained. The intermediates generated from pyridine and fluorinated substrates (1d and 1e) exhibited analogous decomposition profiles as the intermediate generated from substrate 1a but were confirmed to show lower optimal yields of 48% and 67% due to relative instability (FIGS. 1C and 1D). However, it is worth highlighting that other lithiated sulfonyl fluoride intermediates can also be effectively controlled through a capillary microreactor. In addition, the rearrangement of the fluorosulfate intermediate can be controlled under cryogenic temperature and 16 ms conditions in flow.

With the optimized conditions in hand, the inventors focused on the functionalization of benzenesulfonyl fluoride by introducing various electrophiles (FIG. 2). Employing the more reactive methyl triflate as an electrophile instead of iodomethane led to the formation of methylated product 2ab in 80% yield. In addition, products 2ac and 2ad were obtained in yields of 61% and 93% through silylation and stannylation, respectively.

Halogenation and borylation successfully afforded products 2af-2ah in yields of 27% to 94%. In particular interest, the use of electrophiles such as benzaldehyde and phenyl isocyanate induced intra-molecular SuFEx cyclization by attacking their own S—F handles in tandem. It must be pointed out that this cyclization was impossible in traditional batch reactors.

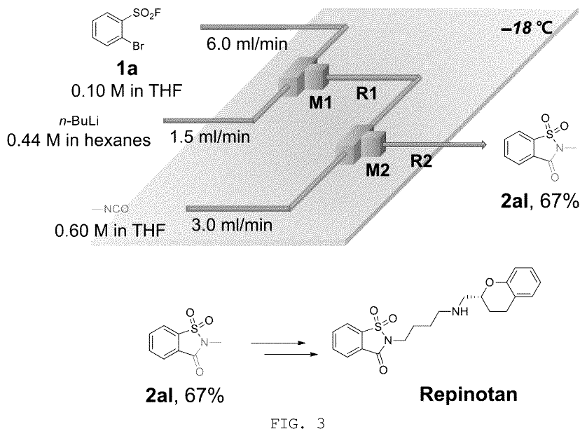

To showcase the utility of this synthetic approach, the inventors embarked on the synthesis of a precursor of the drug repinotan, a selective 5-HT_{1A} receptor full agonist (FIG. 3). The same optimal reaction conditions as in the precedent functionalized sulfonyl fluoride synthesis were employed, and the synthesis of precursor 2al was successfully achieved in a yield of 67%. Notably, this approach rapidly and straightforwardly forms the saccharin structure at −18° C., a remarkable contrast to the reported methods that necessitate high temperatures, extended reaction times, or both and involve two subsequent reactions to obtain repinotan.

Furthermore, the inventors embarked on a continuous-flow-based integrated synthesis that incorporates SuFEx reactions using various organolithium nucleophiles into the aforementioned ultrafast functionalization. This approach leverages the precise control of organolithium reactions and the process robustness offered by the capillary microreactor. Prior to the integrated synthesis, experiments using increased equivalents of n-BuLi and reduced equivalents of tert-butanol were carried out to confirm the feasibility of flow-based SuFEx connection and prevent a competitive side reaction that inhibits the SuFEx reaction of the newly introduced organolithium nucleophiles resulting from the use of an excessive amount of electrophile, respectively.

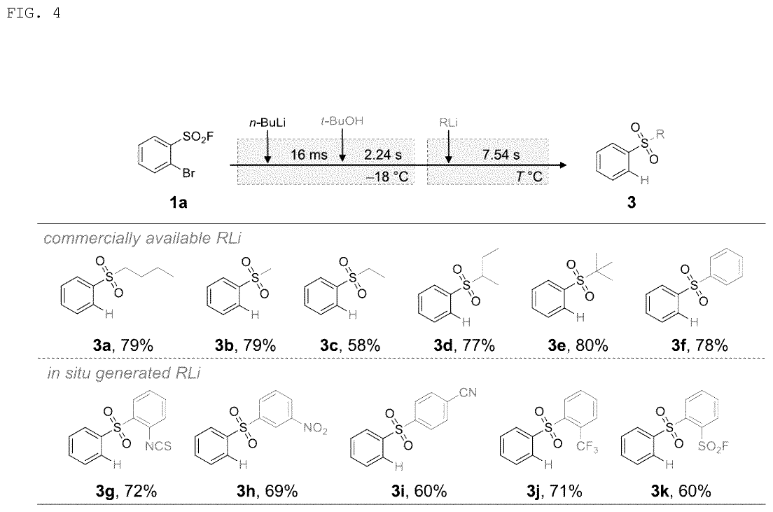

Eventually, the inventors implemented an integrated synthesis that incorporates SuFEx reactions into the control of the aryllithium intermediate using tert-butanol as a general electrophile to construct S—C bonds with organolithium nucleophile modules (FIG. 4). The integrated synthesis was initiated using moderately unstable commercially available alkyl-and aryllithium reagents as nucleophiles through a capillary microreactor. Methyllithium, ethyllithium, each type of butyllithium, and phenyllithium effectively formed S—C bonds, providing sulfone compounds (3a-3f) in high yields (58-80%). However, the SuFEx reaction with ethyllithium, available only as a low-concentration reagent and used as a stock solution, was conducted at room temperature due to the freezing of the reagent at a low temperature such as −18° C., resulting in the desired product in a relatively low yield of 58%.

Furthermore, the inventors anticipated that highly reactive, short-lived aryllithium nucleophiles could also be successfully introduced into the SuFEx reaction by utilizing a capillary microreactor. Thus, a capillary microreactor was employed for these more challenging SuFEx reactions with unstable aryllithium intermediates. The optimal conditions for each aryllithium intermediate were carefully selected to ensure sufficient SuFEx reaction prior to their decompositions, yielding functionalized diphenyl sulfones in high yields (60-72%). Remarkably, the inventors synthesized the product 3k, which involves a sulfonyl fluoride moiety for additional SuFEx connection, by employing the lithiated benzenesulfonyl fluoride intermediate, the primary focus of this study, as a nucleophile.

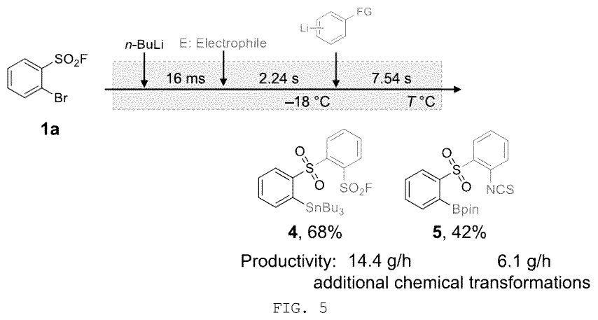

To illustrate the effectiveness of our integrated synthesis approach, the inventors conducted a practical demonstration involving the introduction of a tributyltin or pinacolboranyl group, followed by the SuFEx reaction with aryllithium intermediates primed for further reactions with nucleophiles (FIG. 5). The desired sulfone products 4 and 5 were achieved in yields of 68% and 42%, respectively, under identical reaction conditions employed for the synthesis of 3k and 3g, potentially allowing for the production of approximately 14.4 and 6.1 g/h. It is noteworthy that both terminal functional groups present in products 4 and 5 serve as pivotal building blocks in molecular assembly, providing adaptability for a diverse range of chemical transformations, encompassing coupling and SuFEx reactions.

In conclusion, the inventors achieved ultrafast functionalization of arylsulfonyl fluorides under mild temperature (−18° C.) by precisely controlling the aryllithium intermediate in a capillary microreactor with excellent residence time controllability. This innovative synthetic approach enabled various functionalized sulfonyl fluoride syntheses, including a pivotal precursor of the drug repinotan, in high yields (27-94%) within seconds. Moreover, the inventors seamlessly integrated the continuous-flow-based SuFEx reaction with even unstable organolithium intermediates, resulting in the rapid production of various sulfone products in high yields (42-72%) within a short 10 s time frame. This strategic approach is expected to significantly contribute to the fields of organic synthesis and bioorthogonal chemistry by providing a rapid and efficient means for synthesizing complex functionalized sulfonyl fluorides using a simple, stable, and more manageable sulfonyl fluoride substrate that is capable of molecular assembly with even unstable reactants.

### Example 1

### Example 1-1: Preparation of Bromobenzenesulfonyl Fluoride (1a-c)

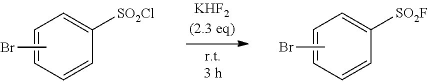

Potassium bifluoride (17.75 g, 227.3 mmol, 2.3 equiv.) was dissolved in H₂O (50 mL) to make a saturated solution. A solution of bromobenzenesulfonyl chloride (25 g in 100 mL MeCN, 0.98 M, 97.8 mmol, 1 equiv.) was added to a prepared saturated solution of potassium bifluoride. The reaction mixture was stirred for 3 h at room temperature and analyzed by GC. The organic phase was separated and the aqueous phase was extracted with ethyl acetate. After the combined organic phase was washed with 10% aqueous NaCl and dried over Na₂SO₄, solvent was removed under reduced pressure.

### Example 1-1-1: 2-Bromobenzenesulfonyl Fluoride (1a)

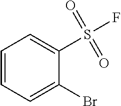 [SMILES: O=S(=O)(F)c1ccccc1Br]

2-Bromobenzenesulfonyl fluoride (1a) was obtained as an off-white solid in 97% yield (16.13 g): ¹H NMR (500 MHz, CDCl₃) δ 8.11 (dd, J=4.8 Hz, 1H), 7.83 (dd, J=4.6 Hz, 1H), 7.59-7.52 (m, 2H) ppm; ¹³C NMR (125 MHz, CDCl₃) δ 136.3, 136.1, 134.2, 134.0, 132.2, 128.2, 121.2 ppm; ¹⁹F NMR (470 MHz, CDCl₃) δ 58.9 ppm. The spectral data were identical to those reported in the literature.

### Example 1-1-2: 3-Bromobenzenesulfonyl Fluoride (1b)

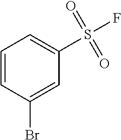 [SMILES: O=S(=O)(F)c1cccc(Br)c1]

3-Bromobenzenesulfonyl fluoride (1b) was obtained as a light yellow oil in 99% yield (16.47 g): ¹H NMR (500 MHz, CDCl₃) δ 8.10 (t, J=1.8 Hz, 1H), 7.92 (dt, J=5.3 Hz, 1H), 7.88 (dt, J=4.9 Hz, 1H), 7.50 (td, J=8.3, 1H) ppm; ¹³C NMR (125 MHz, CDCl₃) δ 138.9, 134.8, 134.6, 131.3 (2C), 127.1, 123.6 ppm; ¹⁹F NMR (470 MHz, CDCl₃) δ 67.2 ppm. The spectral data were identical to those reported in the literature.

### Example 1-1-3: 4-Bromobenzenesulfonyl Fluoride (1c)

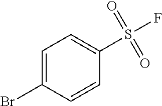 [SMILES: O=S(=O)(F)c1ccc(Br)cc1]

4-Bromobenzenesulfonyl fluoride (1c) was obtained as a white solid in 98% yield (16.30 g): ¹H NMR (500 MHz, CDCl₃) δ 7.86 (dd, J=4.3, 2H), 7.77 (dd, J=4.4 Hz, 2H) ppm; ¹³C NMR (125 MHz, CDCl₃) δ 133.3, 132.3, 132.1, 131.5, 130.0 ppm; ¹⁹F NMR (470 MHz, CDCl₃) δ 67.3 ppm. The spectral data were identical to those reported in the literature.

### Example 1-2: Preparation of 2-Bromo-3-pyridinesulfonyl Fluoride and 2-Bromo-6-fluorobenzenesulfonyl Fluoride (1d and 1e)

(a) Thionyl chloride (42 mL, 0.577 mol, 4.3 equiv.) was added dropwise over 60 min to water (250 mL), cooled to 0° C., maintaining the temperature of the mixture 0-7° C. The solution was allowed to warm to 18° C. over 17 h. Copper(I) chloride (0.151 g, 0.0015 mol, 0.01 equiv.) was added to the mixture, and the resultant yellow-green solution was cooled to −3° C. using an acetone/ice bath.

(b) Hydrochloric acid (36% w/w, 135 mL) was added, with agitation, to 3-amino-2-bromopyridine or 2-bromo-6-fluoroaniline (0.135 mol, 1 equiv.), maintaining the temperature of the mixture below 30° C. with ice cooling. The reaction mixture (1.0 M) was cooled to −5° C. using an ice/acetone bath and a solution of sodium nitrite (10.0 g, 0.145 mol, 1.08 equiv.) in water (40 mL) was added dropwise over 45 min, maintaining the temperature of the reaction mixture between −5° C. to 0° C., the resultant slurry was cooled to −2° C. and stirred for 10 min.

(c) The slurry from step b was cooled to −5° C. and added to the solution obtained from step a over 95 min, maintaining the temperature of the reaction mixture between −3° C. to 0° C. (the slurry from step b was maintained at −5° C. throughout the addition). When the addition was complete, the reaction mixture was agitated at 0° C. for 75 min. The desired product was obtained by extraction by adding ethyl acetate to the reaction mixture, and dried under vacuum at below 35° C.

(d) Sulfonyl fluorides were prepared through the Halex reaction of the sulfonyl chlorides obtained in (c) as in the preparation of 1a-1c.

### Example 1-2-1: 2-Bromo-3-pyridinesulfonyl Fluoride (1d)

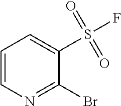 [SMILES: O=S(=O)(F)c1cccnc1Br]

2-Bromo-3-pyridinesulfonyl fluoride (1d) was obtained as a yellow solid in 62% yield (20.01 g) ¹H NMR (500 MHz, CDCl₃) δ 8.67 (dd, J=3.3 Hz, 1H), 8.37 (dd, J=4.9 Hz, 1H), 8.27 (dd, J=3.2 Hz, 1H) ppm; ¹³C NMR (125 MHz, CDCl₃) δ 155.1, 148.1, 142.4, 140.8, 123.5 ppm; ¹⁹F NMR (470 MHz, CDCl₃) δ 59.8 ppm; HRMS (EI) (m/z) C₅H₃BrFNO₂S⁺ [M]⁺: 238.9052; found: 238.9054.

### Example 1-2-2: 2-Bromo-6-fluorobenzenesulfonyl fluoride (1e)

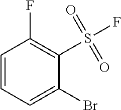 [SMILES: O=S(=O)(F)c1c(F)cccc1Br]

2-Bromo-6-fluorobenzenesulfonyl fluoride (1e) was obtained as a dark brown oil in 68% yield (23.49 g): ¹H NMR (500 MHz, CDCl₃) δ 7.69 (d, J=4.0 Hz, 1H), 7.59 (d, J=10.9 Hz, 1H), 7.33 (q, J=9.5 Hz, 1H) ppm; ¹³C NMR (125 MHz, CDCl₃) δ 161.8, 137.0, 132.0, 127.8, 122.2, 117.5 ppm; ¹⁹F NMR (470 MHz, CDCl₃) δ 69.7, −99.3 ppm; HRMS (EI) (m/z) C₆H₃BrF₂O₂S⁺ [M]⁺: 255.9005; found: 255.9007.

### Example 1-3: Preparation of 2-Bromophenyl Fluorosulfate (1f)

To a 100 mL vial containing the 2-bromophenol (1.07 mL, 10 mmol, 1.0 equiv.) and AISF (3.77 g, 12 mmol, 1.2 equiv.) was added tetrahydrofuran (50 mL) followed by 1,8-diazabicyclo[5.4.0]undec-7-ene (3.29 mL, 22 mmol, 2.2 equiv.) over a period of 390 seconds. The reaction mixture was stirred at room temperature for 130 minutes and then diluted with ethyl acetate or ether and washed with either 0.5 N KHSO₄ or 0.5 N HCl (x2) and brine (x1). The combined organic fraction was dried with anhydrous sodium sulfate and concentrated under reduced pressure. The crude residue was purified by silica gel flash chromatography.

### Example 1-3-1: 2-Bromophenyl Fluorosulfate (1f)

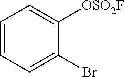

2-Bromophenyl Fluorosulfate (1f) was obtained as a colorless oil in 77% yield (1.96 g). The crude product was extracted and purified by silica gel chromatography (hexane/AcOEt=4/1). The spectral data were identical to those reported in the literature.

### Example 2: Br—Li Exchange Reaction of 2-Bromobenzenesulfonyl Fluoride (1a) Followed by Reaction with Electrophiles in a Capillary Microreactor

A capillary microreactor consisting of two T-shaped micromixers (M1 and M2), two microtube reactors (R1 and R2), and three tube pre-temperature retaining units (P1, P2, and P3 (inner diameter Ø=1000 μm, length L=50 cm)) was used. A 0.10 M solution of a benzenesulfonyl fluoride (1a-1c) in THF (flow rate: 6.0 mL min⁻¹) and a 0.44 M solution of n-BuLi in hexane (flow rate: 1.5 mL min⁻¹) were introduced to M1 (Ø=500 μm) by syringe pumps. The resulting solution was passed through R1 and was mixed with a 0.60 M solution of tert-butanol in THF (flow rate: 3.0 mL min⁻¹) in M2 (Ø=500 μm). The resulting solution was passed through R2 (Ø=1000 μm, L=50 cm). After a steady state was reached, the product solution was collected for 30 s while being quenched with sat. NH₄C1 aqueous solution. The reaction mixture was analyzed by GC. The organic phase was separated and the aqueous phase was extracted with ethyl acetate. After the combined organic phase was dried over Na₂SO₄, solvent was removed. The product was analyzed by ¹H, ¹³C and ¹⁹F NMR and GCMS.

### Example 2-1: 2-Methylbenzenesulfonyl fluoride (2ab)

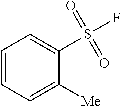 [SMILES: Cc1ccccc1S(=O)(=O)F]

The product 2ab was obtained as a colorless oil in 80% yield (42.7 mg). The crude product was extracted and purified by silica gel chromatography (hexane/AcOEt=4/1): ¹H NMR (500 MHz, CDCl₃) δ 8.02 (dd, J=3.8 Hz, 1H), 7.61 (td, J=7.1 Hz, 1H), 7.41-7.38 (m, 2H), 2.67 (s, 1H) ppm; ¹³C NMR (125 MHz, CDCl₃) δ 139.2, 135.5, 133.0, 130.3, 129.9, 126.8, 20.5 ppm; ¹⁹F NMR (470 MHz, CDCl₃) δ 61.2 ppm. The spectral data were identical to those reported in the literature.

### Example 2-2: 2-(Trimethylsilyl)benzenesulfonyl fluoride (2ac)

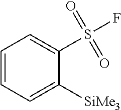 [SMILES: C[Si](C)(C)c1ccccc1S(=O)(=O)F]

The product 2ac was obtained as a white solid in 61% yield (42.4 mg). The crude product was extracted and purified by silica gel chromatography (hexane/AcOEt=4/1): ¹H NMR (500 MHz, CDCl₃) δ 8.13 (dd, J=4.2 Hz, 1H), 7.84 (dd, J=3.8 Hz, 1H), 7.69 (td, J=7.9 Hz, 1H), 7.58 (tt, J=8.0 Hz, 1H), 0.41 (s, 9H) ppm; ¹³C NMR (125 MHz, CDCl₃) δ 142.3, 138.2, 138.0, 137.2, 134.4, 130.5, 129.9, 0.36 ppm; ¹⁹F NMR (470 MHz, CDCl₃) δ 67.6 ppm. The spectral data were identical to those reported in the literature.

### Example 2-3: 2-Tributylstannylbenzenesulfonyl fluoride (2ad)

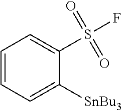

The product 2ad was obtained as a white solid in 93% yield (126.0 mg). The crude product was extracted and purified by silica gel chromatography (hexane/AcOEt=4/1): ¹H NMR (500 MHz, CDCl₃) δ 8.07 (dd, J=8.8 Hz, 1H), 7.73 (dd, J=20.8 Hz, 1H), 7.61 (td, J=7.0 Hz, 1H), 7.50 (td, J=7.6 Hz, 1H), 1.51-1.45 (m, 6H), δ 1.26 (sex, J=10.1 Hz, 6H), δ 1.18-1.11 (m, 6H), δ 0.84 ppm (t, J=7.3 Hz, 9H) ppm; ¹³C NMR (125 MHz, CDCl₃) δ 146.0, 139.5, 139.3, 138.4, 134.0, 129.8, 129.1, 29.0, 27.4, 13.7, 11.6 ppm; ¹⁹F NMR (470 MHz, CDCl₃) δ 67.4 ppm; HRMS (EI) (m/z) C₁₈H₃₁FO₂SSn⁺ [M]⁺: 450.1051; found: 450.1055.

### Example 2-4: Methyl 2-(fluorosulfonyl)benzoate (2ae)

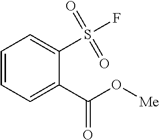 [SMILES: COC(=O)c1ccccc1S(=O)(=O)F]

The product 2ae was obtained as a colorless oil in 63% yield (41.1 mg). The crude product was extracted and purified by silica gel chromatography (hexane/AcOEt=4/1): ¹H NMR (500 MHz, CDCl₃) δ 8.12 (dd, J=4.5 Hz, 1H), 7.84 (dd, J=3.6 Hz, 1H), 7.79 (td, J=8.2 Hz, 1H), 7.70 (tt, J=8.9 Hz, 1H), 3.95 (s, 1H) ppm; ¹³C NMR (125 MHz, CDCl₃) δ 165.9, 135.4, 133.2, 132.2, 131.9, 131.8, 130.9, 130.7, 53.6 ppm; ¹⁹F NMR (470 MHz, CDCl₃) δ 65.4 ppm. The spectral data were identical to those reported in the literature.

### Example 2-5: 2-Iodobenzenesulfonyl fluoride (2af)

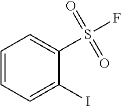 [SMILES: O=S(=O)(F)c1ccccc1I]

The product 2af was obtained as an off-white solid in 94% yield (81.3 mg). The crude product was extracted and purified by silica gel chromatography (hexane/AcOEt=4/1): ¹H NMR (500 MHz, CDCl₃) δ 8.30 (dd, J=4.4 Hz, 1H), 8.16 (dd, J=4.7 Hz, 1H), 7.73 (td, J=7.8 Hz, 1H), 7.57 (td, J=8.4 Hz, 1H) ppm; ¹³C NMR (125 MHz, CDCl₃) δ 143.1, 136.8, 136.1, 136.0, 131.7, 129.2, 94.1 ppm; ¹⁹F NMR (470 MHz, CDCl₃) δ 58.0 ppm. The spectral data were identical to those reported in the literature.

### Example 2-6: 2-Fluorobenzenesulfonyl Fluoride (2Ag)

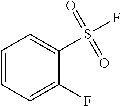 [SMILES: O=S(=O)(F)c1ccccc1F]

The product 2ag was obtained as a colorless oil in 27% yield (13.8 mg). The crude product was extracted and purified by silica gel chromatography (hexane/AcOEt=4/1): ¹H NMR (500 MHz, CDCl₃) δ 8.10 (td, J=8.3 Hz, 1H), 8.04 (td, J=11.4 Hz, 1H), 7.72 (t, J=9.6 Hz, 1H), 7.58 (t, J=7.8 Hz, 1H) ppm; ¹³C NMR (125 MHz, CDCl₃) δ 159.6, 157.5, 139.8, 130.9, 126.1, 118.3, 118.1 ppm; ¹⁹F NMR (470 MHz, CDCl₃) δ 65.9, −107.7 ppm. The spectral data were identical to those reported in the literature.

### Example 2-7: 2-(4,4,5,5-Tetramethyl-1,3,2-dioxaborolan-2-yl)benzenesulfonyl fluoride (2ah)

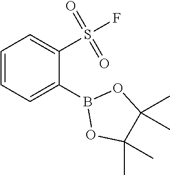 [SMILES: CC1(C)OB(c2ccccc2S(=O)(=O)F)OC1(C)C]

The product 2ah was obtained as a white solid in 47% yield (39.9 mg). The crude product was extracted and purified by silica gel chromatography (hexane/AcOEt=4/1): ¹H NMR (500 MHz, CDCl₃) δ 7.89 (dd, J=5.8 Hz, 1H), 7.63 (t, J=7.4 Hz, 1H), 7.57-7.52 (m, 2H), 1.24 (s, 12H) ppm; ¹³C NMR (125 MHz, CDCl₃) δ 135.7, 134.8, 134.3, 133.8, 130.4, 129.8, 83.0, 24.9 ppm; ¹⁹F NMR (470 MHz, CDCl₃) δ 66.2 ppm; HRMS (EI) (m/z) C₁₂H₁₆BFO₄S⁺ [M]⁺: 286.0846; found: 286.0840.

### Example 2-8: 3-Phenyl-3H-2,1-benzoxathiole 1,1-dioxide (2ai)

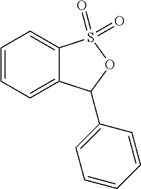 [SMILES: O=S1(=O)OC(c2ccccc2)c2ccccc21]

The product 2ai was obtained as a white solid in 71% yield (52.2 mg). The crude product was extracted and purified by silica gel chromatography (hexane/AcOEt=4/1): ¹H NMR (500 MHz, CDCl₃) δ 7.85 (dd, J=3.6 Hz, 2H), 7.60 (td, J=10.3 Hz, 2H), 7.51 (t, J=7.6 Hz, 2H), 7.40-7.34 (m, 2H), 7.12 (td, J=12.5 Hz, 1H), 7.02 (d, J=3.5 Hz, 1H) ppm; ¹³C NMR (125 MHz, CDCl₃) δ 136.6, 134.7, 130.3, 129.9 (2C), 129.2 (2C), 128.7, 128.6, 128.2, 127.4, 126.3, 108.1 ppm; HRMS (EI) (m/z) C₁₃H₁₀O₃S⁺ [M]⁺: 246.0351; found: 246.0353.

### Example 2-9: 3-(4-Cyanophenyl)-3H-2,1-benzoxathiole 1,1-dioxide (2aj)

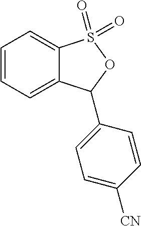 [SMILES: N#Cc1ccc(C2OS(=O)(=O)c3ccccc32)cc1]

The product 2aj was obtained as a white solid in 78% yield (62.7 mg). The crude product was extracted and purified by silica gel chromatography (hexane/AcOEt=4/1): ¹H NMR (500 MHz, CDCl₃) δ 7.86 (dd, J=3.5 Hz, 1H), 7.70 (dd, J=4.1 Hz, 2H), 7.63 (td, J=5.6 Hz, 2H), 7.52 (dd, J=4.1 Hz, 2H), 7.15 (dd, J=4.2 Hz, 1H), 6.60 (s, 1H) ppm; ¹³C NMR (125 MHz, CDCl₃) δ 140.7, 138.9, 137.7, 134.4, 133.2 (2C), 132.4, 130.6, 128.2 (2C), 124.2, 117.9, 114.1, 84.0 ppm; HRMS (EI) (m/z) C₁₄H₉NO₃S⁺ [M]⁺: 271.0303; found: 271.0301.

### Example 2-10: 2-Phenyl-1,2-benzisothiazol-3(2H)-one 1,1-dioxide (2ak)

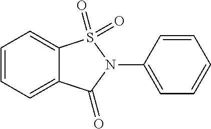 [SMILES: O=C1c2ccccc2S(=O)(=O)N1c1ccccc1]

The product 2ak was obtained as a white solid in 64% yield (49.9 mg). The crude product was extracted and purified by silica gel chromatography (hexane/AcOEt=4/1). The spectral data were identical to those reported in the literature.

### Example 2-11: 2-Methyl-1,2-benzisothiazol-3(2H)-one 1,1-dioxide (2al)

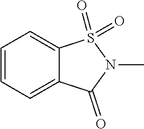

The product 2al was obtained as a white solid in 67% yield (39.8 mg). The crude product was extracted and purified by silica gel chromatography (hexane/AcOEt=4/1). The spectral data were identical to those reported in the literature.

### Comparative Example: Batch System

### Comparative Example 1-1: Methanol Electrophile

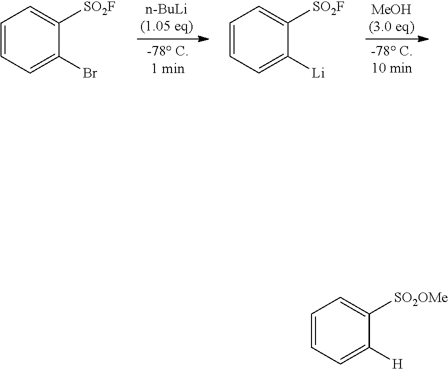

A solution of n-butyllithium (2.5 M in hexanes, 0.42 mL) was added dropwise to a solution of 2-bromobenzenesulfonyl fluoride (0.10 M in THF, 10 mL, 1 mmol) in a 50 mL round bottom glass flask at regular pace with magnetic stirring for 1 min in cooling bath at −78° C. The mixture was stirred for 1 min and methanol (0.12 mL) was added. After stirring for 10 min, cooling bath was removed. The reaction mixture was analyzed by GCMS. Yield was 28%.

### Comparative Example 1-2: Tert-Butanol Electrophile

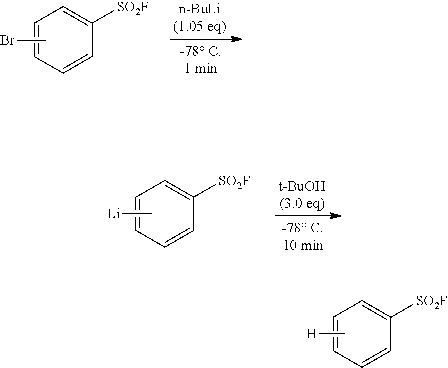

A solution of n-butyllithium (2.5 M in hexanes, 0.42 mL) was added dropwise to a solution of bromobenzenesulfonyl fluoride (0.10 M in THF, 10 mL, 1 mmol) in a 50 mL round bottom glass flask at regular pace with magnetic stirring for 1 min in cooling bath at −78° C. The mixture was stirred for 1 min and tert-butanol (0.29 mL) was added. After stirring for 10 min, cooling bath was removed. The reaction mixture was analyzed by GCMS.

### Comparative Example 1-2-1: Benzenesulfonyl Fluoride (2Aa) with 2-Bromobenzenesulfonyl Fluoride (1a) (Reactant)

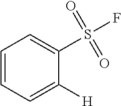

The product 2aa was obtained as a colorless oil in 25% yield (40.2 mg): 1H NMR (500 MHz, CDCl₃) δ 7.97 (dt, J=4.5 Hz, 2H), 7.76 (tt, J=7.5 Hz, 1H), 7.60 (td, J=8.4 Hz, 2H) ppm; 13C NMR (125 MHz, CDCl₃) δ 135.8, 133.2, 133.0, 129.8, 128.5 ppm; ¹⁹F NMR (470 MHz, CDCl₃) δ 66.8 ppm. The spectral data were identical to those reported in the literature. Yield was 25%.

### Comparative Example 1-2-2: 3-Bromobenzenesulfonyl Fluoride (1b) (Reactant)

Except for the use of 3-bromobenzenessulfonyl fluoride instead of 2-bromobenzenesulfonyl fluoride, the Br—Li exchange reaction was followed by tert-Butanol in the batch system in the same manner as comparative example 1-2-1. Benzenesulfonyl fluoride was not detected in the product.

### Comparative Example 1-2-3: 4-Bromobenzenesulfonyl Fluoride (1c) (Reactant)

Except that 4-bromobenzenessulfonyl fluoride was used instead of 2-bromobenzenesulfonyl fluoride, the Br—Li exchange reaction was followed by tert-Butanol in the batch system in the same manner as comparative example 1-2-1. Benzenesulfonyl fluoride was not detected in the product.

### Comparative Example 1-3: 2-bromobenzenesulfonyl chloride (1c) (Reactant)

Except that 2-bromobenzenessulfonyl chloride was used instead of 2-bromobenzenesulfonyl fluoride, the Br—Li exchange reaction was followed by tert-Butanol in the batch system in the same manner as comparative example 1-2-1. Benzenesulfonyl fluoride was not detected in the product

### Example 3: Br—Li Exchange Reaction in Capillary Microreactor Followed by Reaction with Electrophilic Followed by Continuous SuFEx Reaction

### Example 3-1: Use of Commercially Available Organolithium Nucleophiles

FIG. 7A is a procedure for the Br—Li exchange reaction of 2-bromobenzenesulfonyl fluoride (1a) followed by reaction with tert-butanol and subsequent SuFEx reaction with a commercially available organolithium nucleophile in a capillary microreactor.

Referring to FIG. 7A, a capillary microreactor consisting of three T-shaped micromixers (M1, M2 and M3), three microtube reactors (R1, R2 and R3), and four tube pre-temperature retaining units (P1, P2, P3 and P4 (inner diameter Ø=1000 μm, length L=50 cm)) was used. A 0.10 M solution of a 2-benzenesulfonyl fluoride (1a) in THF (flow rate: 6.0 mL min⁻¹) and a 0.44 M solution of n-BuLi in hexane (flow rate: 1.5 mL min⁻¹) were introduced to M1 (Ø=500 μm) by syringe pumps. The resulting solution was passed through R1 and was mixed with a 0.24 M solution of tert-butanol in THF (flow rate: 3.0 mL min⁻¹) in M2 (Ø=500 μm). The resulting solution was passed through R2 (Ø=1000 μm, L=50 cm) and was mixed with a 0.6 M solution of commercially available organolithium in hexane (or Et₂O for MeLi and PhLi, neat for EtLi) (flow rate: 2.0 mL min-1 (or 3.243 mL min-1 for EtLi)) in M3 (Ø=500 μm). The resulting solution was passed through R3 (Ø=1000 μm, L=200 cm). After a steady state was reached, the product solution was collected for 30 s while being quenched with sat. NH₄Cl aqueous solution. The reaction mixture was analyzed by GC. The organic phase was separated and the aqueous phase was extracted with ethyl acetate. After the combined organic phase was dried over Na₂SO₄, solvent was removed. The product was analyzed by ¹H, ¹³C and ¹⁹F NMR and GCMS.

### Example 3-1-1: (Butylsulfonyl)benzene (3a)

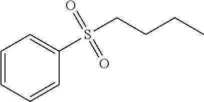

The product 3a was obtained as a colorless oil in 79% yield (47.0 mg). The crude product was extracted and purified by silica gel chromatography (hexane/AcOEt=4/1): ¹H NMR (500 MHz, CDCl₃) δ 7.87 (dt, J=5.0 Hz, 2H), 7.62 (tt, J=8.6 Hz, 1H), 7.53 (td, J=7.5 Hz, 2H), 3.05 (t, J=8.2 Hz, 2H), 1.68-1.62 (m, 2H), 1.39-1.31 (m, 2H), 0.85 (t, J=7.4 Hz, 3H) ppm; ¹³C NMR (125 MHz, CDCl₃) δ 139.4, 133.8, 129.4, 128.2, 56.2, 24.8, 21.7, 13.6 ppm. The spectral data were identical to those reported in the literature.

### Example 3-1-2: (Methylsulfonyl)benzene (3b)

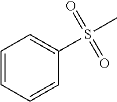

The product 3b was obtained as a white solid in 79% yield (36.8 mg). The crude product was extracted and purified by silica gel chromatography (hexane/AcOEt=4/1): ¹H NMR (500 MHz, CDCl₃) δ 7.88 (dt, J=5.2 Hz, 2H), 7.60 (tt, J=7.6 Hz, 1H), 7.52 (td, J=7.7 Hz, 2H), 3.00 (s, 3H) ppm; ¹³C NMR (125 MHz, CDCl₃) δ 140.6, 133.8, 129.5, 127.4, 44.5 ppm. The spectral data were identical to those reported in the literature.

### Example 3-1-3: (Ethylsulfonyl)benzene (3c)

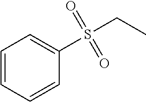

The product 3c was obtained as a colorless oil in 58% yield (30.2 mg). The crude product was extracted and purified by silica gel chromatography (hexane/AcOEt=4/1): ¹H NMR (500 MHz, CDCl₃) δ 7.83 (dt, J=4.0 Hz, 2H), 7.58 (tt, J=7.8 Hz, 1H), 7.50 (td, J=7.7 Hz, 2H), 3.05 (q, J=11.1 Hz, 2H), 1.18 (t, J=7.4 Hz, 3H) ppm; ¹³C NMR (125 MHz, CDCl₃) δ 138.5, 133.7, 129.3, 128.2, 50.6, 7.4 ppm. The spectral data were identical to those reported in the literature.

### Example 3-1-4: ((1-Methylpropyl)sulfonyl)benzene (3d)

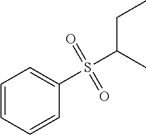

The product 3d was obtained as a colorless oil in 77% yield (46.4 mg). The crude product was extracted and purified by silica gel chromatography (hexane/AcOEt=4/1): ¹H NMR (500 MHz, CDCl₃) δ 7.79 (dt, J=4.9 Hz, 2H), 7.57 (tt, J=8.6 Hz, 1H), 7.48 (td, J=7.5 Hz, 2H), 2.91-2.84 (m, 1H), 1.96-1.88 (m, 1H), 1.39-1.29 (m, 1H), 1.17 (d, J=3.5 Hz, 3H) 0.89 (t, J=7.5 Hz, 3H) ppm; ¹³C NMR (125 MHz, CDCl₃) δ 137.4, 133.6, 129.1, 128.9, 61.5, 22.5, 12.6, 11.1 ppm. The spectral data were identical to those reported in the literature.

### Example 3-1-5: ((1,1-Dimethylethyl)sulfonyl)benzene (3e)

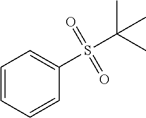

The product 3e was obtained as a white solid in 80% yield (48.2 mg). The crude product was extracted and purified by silica gel chromatography (hexane/AcOEt=4/1): ¹H NMR (500 MHz, CDCl₃) δ 7.85 (dt, J=5.3 Hz, 2H), 7.62 (tt, J=8.8 Hz, 1H), 7.52 (td, J=8.6 Hz, 2H), 1.30 (s, 9H) ppm; ¹³C NMR (125 MHz, CDCl₃) δ 135.5, 133.7, 130.6, 128.9, 59.9, 23.8 ppm. The spectral data were identical to those reported in the literature.

### Example 3-1-6: 1,1′-Sulfonylbisbenzene (3f)

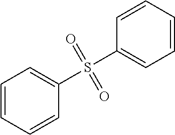 [SMILES: O=S(=O)(c1ccccc1)c1ccccc1]

The product 3f was obtained as a white solid in 78% yield (50.6 mg). The crude product was extracted and purified by silica gel chromatography (hexane/AcOEt=4/1): ¹H NMR (500 MHz, CDCl₃) δ 7.90 (dt, J=4.9 Hz, 4H), 7.49 (tt, J=9.3 Hz, 2H), 7.43 (td, J=8.9 Hz, 4H) ppm; ¹³C NMR (125 MHz, CDCl₃) δ 141.6, 133.3, 129.4, 127.6 ppm. The spectral data were identical to those reported in the literature.

### Example 3-2: Br—Li Exchange Reaction of 2-Bromobenzenesulfonyl Fluoride (1a) Followed by Reaction with Electrophiles and Subsequent SuFEx Reaction with In Situ Generated Organolithium Nucleophile in a Capillary Microreactor

FIG. 7B is a procedure for the Br—Li exchange reaction of 2-bromobenzenesulfonyl fluoride (1a) followed by reaction with an electrophile and subsequent SuFEx reaction with an organolithium nucleophile generated in-situ in a capillary microreactor, FIG. 8 shows an optical image of a capillary microreactor consisting of micromixers (M1-M4) and microtube reactors (R1-R4) for aryllithium reaction and SuFEx coupling using on-site generated organolithium nucleophiles are shown.

Referring to FIGS. 7B and 8, a capillary microreactor consisting of four T-shaped micromixers (M1, M2, M3 and M4), four microtube reactors (R1, R2, R3 and R4), and five tube pre-temperature retaining units (P1, P2, P3, P4 and P5 (inner diameter Ø=1000 μm, length L=50 cm)) was used. A 0.10 M solution of a 2-benzenesulfonyl fluoride (1a) in THF (flow rate: 6.0 mL min⁻¹) and a 0.44 M solution of n-BuLi in hexane (flow rate: 1.5 mL min-were introduced to M1 (Ø=500 μm) by syringe pumps. The resulting solution was passed through R1 and was mixed with a 0.24 M solution of electrophile in THF (flow rate: 3.0 mL min⁻¹) in M2 (Ø=500 μm). The resulting solution was passed through R2 (Ø=1000 μm, L=50 cm). A 0.20 M solution of a functionalized bromobenzene in THF (flow rate: 6.0 mL min⁻¹) and a 0.84 M (or 0.88 M for 1a) solution of n-BuLi (or PhLi for 1-iodo-3-nitrobenzne) in hexane (or Et₂O for PhLi) (flow rate: 1.5 mL min⁻¹) were introduced to M3 (Ø=500 μm) by syringe pumps. The resulting solution was passed through R3 and was mixed with the resulting solution comes from R2 in M4 (Ø=500 μm). The resulting solution was passed through R4 (Ø=1000 μm, L=288 cm). After a steady state was reached, the product solution was collected for 30 s while being quenched with sat. NH4Cl aqueous solution. The reaction mixture was analyzed by GC. The organic phase was separated and the aqueous phase was extracted with ethyl acetate. After the combined organic phase was dried over Na₂SO₄, solvent was removed. The product was analyzed by ¹H, ¹³C and ¹⁹F NMR and GCMS.

### Example 3-2-1: (2-Isothiocyanatophenyl)-sulfonylbenzene (3g)

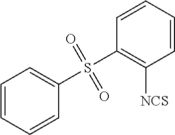

The product 3g was obtained as an off-white solid in 72% yield (59.2 mg). The crude product was extracted and purified by silica gel chromatography (hexane/AcOEt=4/1): ¹H NMR (500 MHz, CDCl₃) δ 8.19-7.76 (m, 2H), 7.66-7.13 (m, 7H) ppm; ¹³C NMR (125 MHz, CDCl₃) δ 136.6, 134.0, 133.3, 133.1, 130.3 (2C), 129.0, 127.8, 126.9, 125.7, 125.2 (2C), 124.0 ppm; HRMS (EI) (m/z) C₁₃H₉NO₂S₂⁺ [M]⁺: 275.0075; found: 275.0079.

### Example 3-2-2: (3-Nitrophenyl)-sulfonylbenzene (3h)

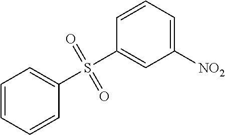 [SMILES: O=[N+]([O-])c1cccc(S(=O)(=O)c2ccccc2)c1]

The product 3h was obtained as a yellow solid in 69% yield (53.8 mg). The crude product was extracted and purified by silica gel chromatography (hexane/AcOEt=4/1): ¹H NMR (500 MHz, CDCl₃) δ 8.21 (td, J=22.4 Hz, 1H), 7.93 (d, J=3.7 Hz, 1H), 7.67 (d, J=3.7 Hz, 3H), 7.55-7.46 (m, 4H) ppm; ¹³C NMR (125 MHz, CDCl₃) δ 148.5, 144.1, 140.3, 134.7, 134.2 (2C), 130.9, 129.8 (2C), 129.4, 128.1 (2C), 123.0 ppm. The spectral data were identical to those reported in the literature.

### Example 3-2-3: (4-Cyanophenyl)-sulfonylbenzene (3i)

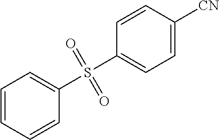 [SMILES: N#Cc1ccc(S(=O)(=O)c2ccccc2)cc1]

The product 3i was obtained as a yellow solid in 60% yield (43.8 mg). The crude product was extracted and purified by silica gel chromatography (hexane/AcOEt=4/1): ¹H NMR (500 MHz, CDCl₃) δ 8.03-7.87 (m, 2H), 7.76 (t, J=8.6 Hz, 1H), 7.67-7.43 (m, 6H) ppm; ¹³C NMR (125 MHz, CDCl₃) δ 146.0, 140.3, 133.8, 133.2 (2C), 129.8, 129.3 (2C), 128.4 (2C), 118.2, 117.3 ppm. The spectral data were identical to those reported in the literature.

### Example 3-2-4: 1-Phenylsulfonyl-2-trifluoromethylbenzene (3j)

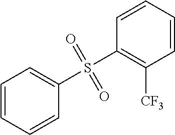 [SMILES: O=S(=O)(c1ccccc1)c1ccccc1C(F)(F)F]

The product 3j was obtained as an off-white solid in 71% yield (61.1 mg). The crude product was extracted and purified by silica gel chromatography (hexane/AcOEt=4/1): ¹H NMR (500 MHz, CDCl₃) δ 8.00 (d, J=3.8 Hz, 1H), 7.94-7.82 (m, 2H), 7.76 (t, J=7.5 Hz, 1H), 7.72-7.67 (m, 1H), 7.63-7.53 (m, 3H), 7.48 (t, J=7.9 Hz, 1H) ppm; ¹³C NMR (125 MHz, CDCl₃) δ 141.4, 140.0, 134.7, 133.7, 133.5, 133.4, 133.2, 132.7, 132.6, 129.7, 129.3, 128.7, 128.7 ppm; ¹⁹F NMR (470 MHz, CDCl₃) δ −55.7 ppm. The spectral data were identical to those reported in the literature.

### Example 3-2-5: 2-Phenylsulfonylbenzenesulfonyl fluoride (3k)

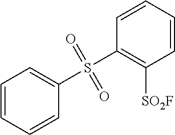 [SMILES: O=S(=O)(F)c1ccccc1S(=O)(=O)c1ccccc1]

The product 3k was obtained as a white solid in 60% yield (53.5 mg). The crude product was extracted and purified by silica gel chromatography (hexane/AcOEt=4/1): ¹H NMR (500 MHz, CDCl₃) δ 8.11 (dd, J=4.8 Hz, 1H), 7.93 (dd, J=5.0 Hz, 2H), 7.82 (dd, J=4.6 Hz, 1H), 7.67 (tt, J=7.5 Hz, 1H), 7.55 (td, J=10.3 Hz, 4H) ppm; ¹³C NMR (125 MHz, CDCl₃) δ 134.1, 133.8, 133.4, 133.2, 133.0, 129.8, 129.3, 129.2, 128.7 (2C), 128.2 (2C) ppm; ¹⁹F NMR (470 MHz, CDCl₃) δ 67.5 ppm; HRMS (EI) (m/z) C1₂H₉FO₄S₂⁺ [M]⁺: 299.9926; found: 299.9924.

### Example 4: 2-(2-Tributylstannylphenyl)-sulfonylbenzenesulfonyl fluoride (4)

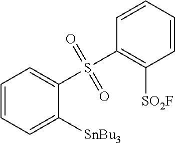

The product 4 was obtained as a white solid in 68% yield (120.3 mg). The crude product was extracted and purified by silica gel chromatography (hexane/AcOEt=4/1): ¹H NMR (500 MHz, CDCl₃) δ 8.09 (dd, J=8.7 Hz, 1H), 8.00 (dd, J=4.9 Hz, 1H), 7.94-7.88 (m, 1H), 7.73 (t, J=6.6 Hz, 1H), 7.62 (tt, J=7.5 Hz, 2H), 7.56-7.50 (m, 2H), 1.51-1.45 (m, 6H), δ 1.30 (sex, J=18.3 Hz, 6H), δ 1.16-1.13 (m, 6H), δ 0.85 ppm (t, J=7.3 Hz, 9H) ppm; ¹³C NMR (125 MHz, CDCl₃) 146.2, 139.4, 138.5, 135.7, 134.7, 134.0, 129.9 (2C), 129.8, 129.3, 129.1, 128.6, 29.1, 27.5, 13.8, 11.7 ppm; ¹⁹F NMR (470 MHz, CDCl₃) δ 67.4 ppm; HRMS (EI) (m/z) C₂₄H₃₅FO₄S₂Sn⁺ [M]⁺: 590.0983; found: 590.0985.

### Example 5: 4,4,5,5-Tetramethyl-2-[2-[(2-isothiocyanatophenyl)sulfonyl]phenyl]-1,3,2-dioxaborolane (5)

The product 5 was obtained as an off-white solid in 42% yield (51.2 mg).

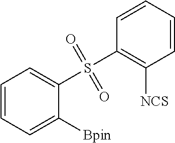

4,4,5,5-Tetramethyl-2-[2-[(2-isothiocyanatophenyl)sulfonyl]phenyl]-1,3,2-dioxaborolane (5). The crude product was extracted and purified by silica gel chromatography (hexane/AcOEt=4/1): ¹H NMR (500 MHz, CDCl₃) δ 7.98 (d, J=3.7 Hz, 1H), 7.92 (dd, J=4.0 Hz, 1H), 7.83 (t, J=7.7 Hz, 1H), 7.50-7.40 (m, 5H), 1.21 (s, 12H) ppm; ¹³C NMR (125 MHz, CDCl₃) δ 137.2, 136.8, 135.5, 134.7, 133.9, 132.8, 131.4, 129.8, 129.6, 128.8, 128.4, 126.7, 84.9, 24.9 ppm; HRMS (EI) (m/z) C₁₉H₂₀BNO₄S₂⁺ [M]⁺: 401.0927; found: 401.0929.

### Experimental Example

### Experimental Example 1: Br—Li Exchange Reaction of 2-Bromobenzenessulfonyl Fluoride (1a) and n-BuLi Followed by Reaction with the Electrophile in Batch System

Table 1 summarizes the reaction yield with the electrophile in the batch systems of comparative examples 1-1, 1-2-1, 1-2-2, 1-2-3, and 1-3.

TABLE 1

| category | Reactants | Electrophilic | Yield (%) |
|---|---|---|---|
| comparative | 2- | Methanol | 28 |
| example | bromobenzenesulfonyl | | |
| 1-1 | fluoride(1a) | | |
| comparative | 2- | tert- | 25 |
| example | bromobenzenesulfonyl | Butanol | |
| 1-2-1 | fluoride(1a) | | |
| comparative | 3- | tert- | non- |
| example | bromobenzenesulfonyl | Butanol | detection |
| 1-2-2 | fluoride(1b) | | (n.d.) |
| comparative | 4- | tert- | non- |
| example | bromobenzenesulfonyl | Butanol | detection |
| 1-2-3 | fluoride(1c) | | (n.d.) |
| comparative | Bromobenzenesulfonyl | tert- | — |
| example | chloride | Butanol | |
| 1-3 | | | |

Referring to Table 1, the inventors observed an undesired product where the sulfonyl fluoride group was attacked by the in situ-generated nucleophile, methoxide ion, when methanol was used as the electrophilic trapping reagent. To prevent this undesired nucleophilic attack on the sulfonyl fluoride group by leveraging the steric hindrance of the tert-butoxide ion, tert-butanol was utilized as an electrophile instead of methanol, leading to the desired protonated product in 25% yield. Meanwhile, the desired product was not detected in the feasibility experiments involving substrates 1b and 1c bearing a sulfonyl fluoride group at the meta or para position, respectively, due to the higher instability compared to the o-lithiated intermediate stabilized through cyclic chelation of a five-membered ring. Additionally, the feasibility study on aryllithium species bearing a sulfonyl chloride group revealed low controllability of the intermediate due to the lower stability of the S—Cl bond.

### Experimental Example 2: Br—Li Exchange Reaction of 2-Bromobenzenessulfonyl Fluoride (1a) and n-BuLi Followed by Reaction with the Electrophilic

FIG. 1A is a schematic diagram of the synthesis method of the ortho-functionalized benzenesulfonyl fluoride derivative in a capillary microreactor and the effect of temperature and residence time of R1 (t^{R1}) and R2 (t^{R2}).

Referring to FIG. 1A, based on the above results, the inventors carried out Br—Li exchange and trapped the intermediate with an electrophile using a capillary microreactor to precisely control the intermediate. In our endeavor to enhance subsequent reactions of the generated intermediate with electrophiles, the inventors meticulously screened residence times and temperatures to investigate their effects on the yield, as determined by ¹H NMR spectroscopy. The o-lithiated benzenesulfonyl fluoride intermediate exhibited increased decomposition with prolonged residence time in R1, except when screened at −58° C. Conversely, under the −58° C. condition, yields were increased with elongated residence time in R1, probably because increased Br—Li exchange progress outweighed the intermediate decomposition. Multiple screenings of the entire conditions led to the identification of optimal conditions (t^{R1}=0.016 s, T=−18° C.) that provided the highest yield of 82%. It is noteworthy that the presented synthetic methodology afforded the desired sulfonyl fluoride within seconds under mild temperatures as well as in dramatically improved yields.

### Experimental Example 3: Confirmation of Functionalized Sulfonyl Fluoride Synthesis by Controlling Aryllithium Intermediates Under Optimal Conditions in Capillary Microreactor

FIG. 2 is a synthesis of functionalized sulfonyl fluoride through the preparation of aryl lithium intermediates under optimal conditions in a capillary microreactor.

Referring to FIG. 2, With the optimized conditions in hand, the inventors focused on the functionalization of benzenesulfonyl fluoride by introducing various electrophiles. Employing the more reactive methyl triflate as an electrophile instead of iodomethane led to the formation of methylated product 2ab in 80% yield. In addition, products 2ac and 2ad were obtained in yields of 61% and 93% through silylation and stannylation, respectively. Halogenation and borylation successfully afforded products 2af-ah in yields of 27% to 94%. In particular interest, the use of electrophiles such as benzaldehyde and phenyl isocyanate induced intra-molecular SuFEx cyclization by attacking their own S—F handles in tandem. It must be pointed out that this cyclization was impossible in traditional batch reactors (Comparative example).

### Experimental Example 4: Confirmation of Repinotan Precursor Synthesis

FIG. 3 is a control of aryllithium intermediates in a capillary microreactor demonstrated the synthesis of repinotan precursors as a neuroprotective drug to reduce brain damage after drugs for external wound of head.

Referring to FIG. 3, to showcase the utility of this synthetic approach, the inventors embarked on the synthesis of a precursor of the drug repinotan, a selective 5-HT_{1A} receptor full agonist. The same optimal reaction conditions as in the precedent functionalized sulfonyl fluoride synthesis were employed, and the synthesis of precursor 2al was successfully achieved in a yield of 67%. Notably, this approach rapidly and straightforwardly forms the saccharin structure at −18° C., a remarkable contrast to the reported methods that necessitate high temperatures, extended reaction times, or both and involve two subsequent reactions to obtain repinotan.

### Experimental Example 5: One-Flow Integrated Synthesis Including Ultrafast Functionalization of Aryl Sulfonyl Fluoride and SuFEx Reactions with Various Organolithium Nucleophiles

FIG. 4 shows a one-flow integrated synthesis including ultra-fast functionalization of aryl sulfonyl fluoride and SuFEx reaction with various organolithium nucleophiles.

Referring to FIG. 4, the inventors implemented an integrated synthesis that incorporates SuFEx reactions into the control of the aryllithium intermediate using tert-butanol as a general electrophile to construct S—C bonds with organolithium nucleophile modules through a capillary microreactor consisting of three T-shaped micromixers (M1, M2 and M3) and three microtubes (R1, R2 and R3). The integrated synthesis was initiated using moderately unstable commercially available alkyl- and aryllithium reagents as nucleophiles through a capillary microreactor. Methyllithium, ethyllithium, each type of butyllithium, and phenyllithium effectively formed S—C bonds, providing sulfone compounds (3a-3f) in high yields (58-80%). However, the SuFEx reaction with ethyllithium, available only as a low-concentration reagent and used as a stock solution, was conducted at room temperature due to the freezing of the reagent at a low temperature such as −18° C., resulting in the desired product in a relatively low yield of 58%.

Furthermore, the inventors anticipated that highly reactive, short-lived aryllithium nucleophiles could also be successfully introduced into the SuFEx reaction by utilizing a capillary microreactor. Thus, a capillary microreactor consisting of 4 T-shaped micromixers (M1, M2, M3 and M4) and 4 microtube reactors (R1, R2, R3 and R4) was employed for these more challenging SuFEx reactions with unstable aryllithium intermediates. The optimal conditions for each aryllithium intermediate were carefully selected to ensure sufficient SuFEx reaction prior to their decompositions, yielding functionalized diphenyl sulfones in high yields (60-72%). Remarkably, the inventors synthesized the product 3k, which involves a sulfonyl fluoride moiety for additional SuFEx connection, by employing the lithiated benzenesulfonyl fluoride intermediate, the primary focus of this study, as a nucleophile.

### Experimental Example 6: Confirmation of One-Flow Integrated Synthesis of Sulfones Containing Tributylthin and Sulfonyl Fluoride Groups

FIG. 5 shows a one-flow integrated synthesis of sulfones (4) containing tributylthin and sulfonyl fluoride groups for further chemical transformation.

Referring to FIG. 5, to illustrate the effectiveness of our integrated synthesis approach, the inventors conducted a practical demonstration involving the introduction of a tributyltin or pinacolboranyl group, followed by the SuFEx reaction with aryllithium intermediates primed for further reactions with nucleophiles. The desired sulfone products 4 and 5 were achieved in yields of 68% and 42%, respectively, under identical reaction conditions employed for the synthesis of 3k and 3g, potentially allowing for the production of approximately 14.4 and 6.1 g/h. It is noteworthy that both terminal functional groups present in products 4 and 5 serve as pivotal building blocks in molecular assembly, providing adaptability for a diverse range of chemical transformations, encompassing coupling and SuFEx reactions.

### Experimental Example 7: Control of Product Selectivity by Controlling Concentration

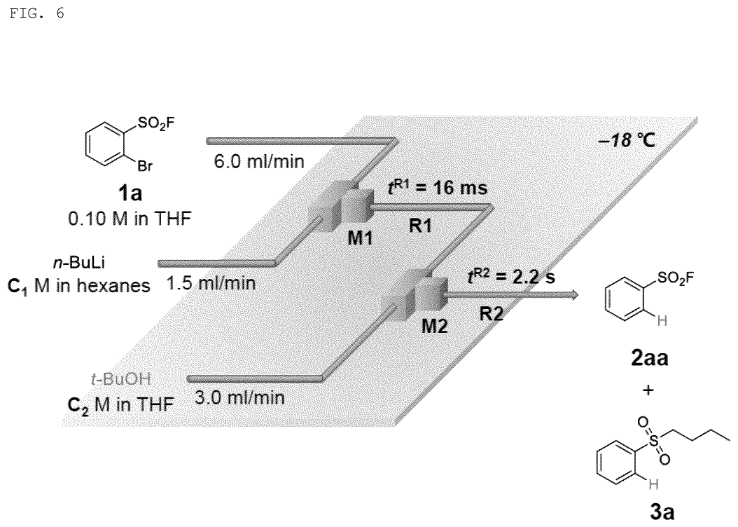

FIG. 6 and Table 2 show an effect of increasing n-BuLi equivalents and decreasing t-BuOH equivalents on the yields of benzenesulfonyl fluoride (2a) and SuFEx product (3a).

TABLE 2

| | C₁ | C₂ | Yield of Example 2 | Yield of Example 3-1-1 |
|---|---|---|---|---|
| Entry | [M] | [M] | Yield | Yield |
| 1 | 0.44 | 0.6 | 82 | n.d. |
| 2 | 0.66 | 0.6 | 61 | 23 |
| 3 | 0.88 | 0.6 | 45 | 33 |
| 4 | 0.44 | 0.4 | 79 | n.d. |
| 5 | 0.44 | 0.3 | 78 | n.d. |
| 6 | 0.44 | 0.24 | 77 | n.d. |

In the Table 2, C₁=0.44, 0.66, and 0.88 M for 1, 1, 1.65, and 2.2 equivalent of n-BuLi, respectively. C₂=0.6, 0.4, 0.3, and 0.24 for 3.0, 2.0, 1.5, and 1.2 equivalent of t-BuOH, respectively. Determined by ¹H NMR spectroscopy using 1,3,5-trimethoxybenzene as an internal standard.

Referring to FIG. 6 and Table 2, the increased equivalents of n-BuLi resulted in a decreased yield of product 2a (Example 2) and an increased yield of the sulfone compound 3a (Example 3-1-1 (entry 1-3)). This result indicates the regioselectivity of n-BuLi as the primary reactant in the Br—Li exchange reaction, followed by the engagement of residual quantities in nucleophilic attacks on the sulfonyl fluoride group. The desired product 2a was obtained in a yield of 77%, which was not significantly reduced even when the equivalent of the tert-butanol was decreased from 3.0 to 1.2 (entry 4-6).

In conclusion, the ultra-fast functionalization of aryl sulfonyl fluoride at a mild temperature (−18° C.) was successfully achieved by precise control of the intersection of aryllithium containing the sulfonyl fluoride group in a capillary microreactor with excellent residence time control within a reaction time of less than 1 second. This innovative synthesis approach made it possible to synthesize a wide range of functionalized sulfonyl fluorides, including pivotal precursors of the drug lepinotan, within seconds with high yields (61˜93%). By effectively minimizing the prevailing side reactions common in batch systems and using sequential lithiation-capture methods, our approach has proven to be exceptionally efficient. In addition, by seamlessly integrating the continuous flow-based SuFEx reaction into the functionalization process mentioned above, it was possible to quickly produce a variety of sulfone products with high yields (60˜72%) within a short 10 seconds. This process was achieved by introducing commercially available or field-generated, highly reactive organolithium reactants for the SuFEx reaction. This strategic approach is expected to make a significant contribution to the field of organic synthesis and bioorthogonal chemistry by providing a rapid and efficient means for the synthesis of complex functionalized sulfonyl fluoride using a simple, stable and non-toxic sulfonyl fluoride substrate. Even when dealing with unstable reactants, more complex compounds can be prepared through molecular assembly.

The scope of the present disclosure is defined by the following claims rather than the above detailed description, and all changes or modifications derived from the meaning and scope of the claims and their equivalent concepts should be interpreted as falling into the scope of the present disclosure.

What is claimed is:

1. A method of synthesizing an ortho-functionalized benzenesulfonyl fluoride derivative, the method comprising:
(a) reacting a benzenesulfonyl fluoride derivative represented by structural formula 1 with an organolithium represented by structural formula 2, thus synthesizing an intermediate represented by structural formula 3 in reaction scheme 1; and
(b) reacting the intermediate with an electrophile, thus synthesizing a compound represented by structural formula 4 or a compound represented by structural formula 4′. 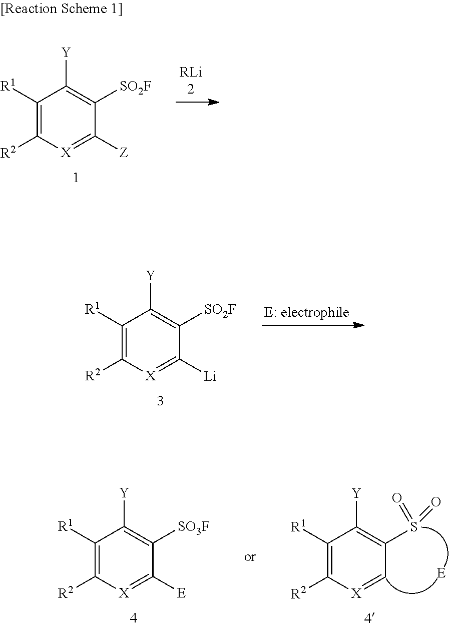
in the reaction scheme 1, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group,
X is a carbon atom or a nitrogen atom,
Y is a hydrogen atom, a fluorine atom, a chlorine atom, a bromine atom, or an iodine atom,
Z is a fluorine atom, a chlorine atom, a bromine atom, or an iodine atom,
R is C1 to C9 alkyl group of or C6 to C14 aryl group, and
E is an electrophile.

2. The method of claim 1, wherein the method is an ultra-fast synthesizing method using ultra-fast flow of the reactants.

3. The method of claim 1, wherein the electrophile is substituted with lithium at the location of ortho position of the sulfonyl fluoride group of the intermediate, or the electrophile is substituted with lithium at the location of ortho position of the sulfonyl fluoride of the intermediate and is substituted with fluorine atom of the sulfonyl fluoride, thus an intra-molecular cyclization reaction being carried out.

4. The method of claim 1, wherein the method is carried out in a flow-based capillary microreactor.

5. The method of claim 1, wherein the step (a) is carried out for a duration of 0.016 to 6.3 seconds, and the step (b) is carried out for a duration of 1.5 to 3 seconds.

6. The method of claim 1, wherein the electrophile of the step (b) comprises at least one selected from the group consisting of t-BuOH, methyl triflate, chlorotrimethylsilane, tributyltin chloride, methyl chloroformate, iodine, N-fluorobenzenesulfonimide, isopropoxy-4,4,5,5-tetramethyl-1,3,2-dioxaborolane, benzaldehyde, 4-formylbenzonitrile, phenyl isocyanate, and methyl isocyanate.

7. The method of claim 1, wherein the product of step (b) comprises at least one selected from the group consisting of

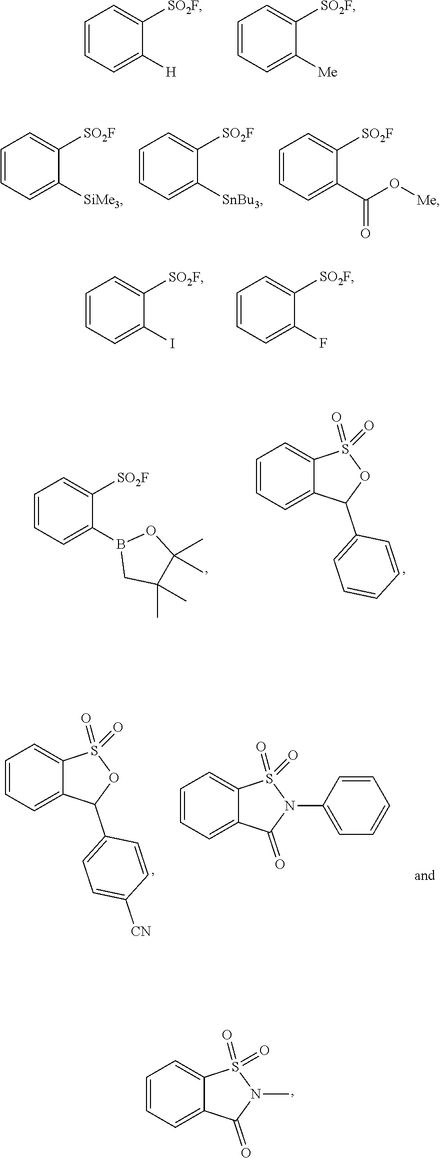

8. The method of claim 1, wherein the step (a) and step (b) are each independently carried out at a temperature in a range of −58 to 25° C.

9. The method of claim 1, wherein the method further comprises:
(c) in reaction scheme 2, the compound represented by structural formula 4 is reacted with a compound represented by structural formula 5 to synthesize a compound represented by structural formula 6,
wherein the step (c) is carried out after the step (b). 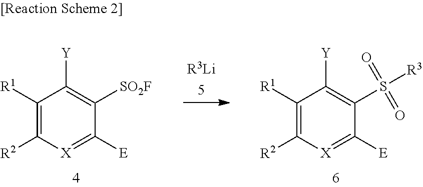
in the reaction scheme 2, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group,
X is a carbon atom or a nitrogen atom,
Y is a hydrogen atom, a fluorine atom, a chlorine atom, a bromine atom, or an iodine atom,
R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and
E is an electrophile.

10. The method of claim 9, wherein the compound represented by the structural formula 5 comprises at least one selected from the group consisting of

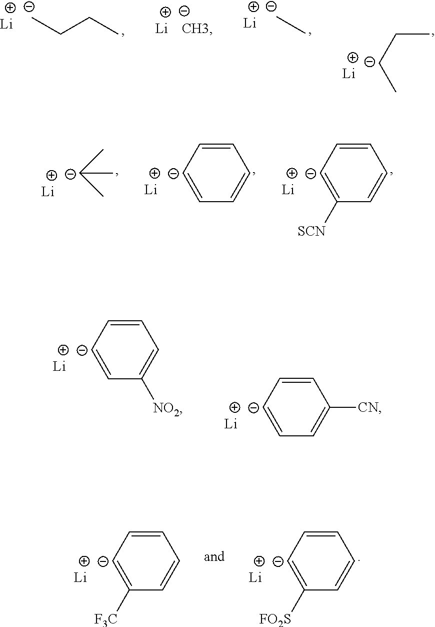

11. The method of claim 9, wherein the compounds represented by the structural formula 6 comprises at least one selected from the group consisting of

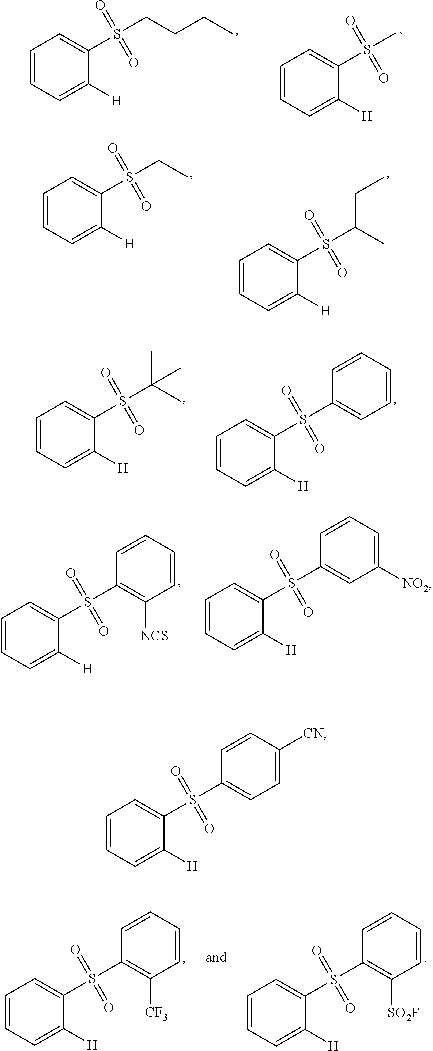

12. The method of claim 9, wherein the method further comprises:
(a′) reacting a compound represented by structural formula 5a with a compound represented by structural formula 5b, thus synthesizing a compound represented by structural formula 5 in reaction scheme 3, wherein the step (a′) is carried out before the step (c). 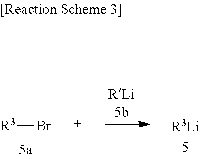
in the reaction scheme 3, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and
R′ is C1 to C9 alkyl group or C6 to C14 aryl group.

13. An ultra-fast synthesis apparatus of an ortho-functionalized benzenesulfonyl fluoride derivative, the apparatus comprising:
a first micromixer M1 which prepares a first mixture by mixing a benzenesulfonyl fluoride derivative represented by Formula 1 and an organolithium represented by structural formula 2 in reaction scheme 1;
a first microreactor R1 which receives the first mixture from the first micromixer M1 and reacts it to synthesize an intermediate represented by structural formula 3;
a second micromixer M2 which prepare a second mixture by mixing the intermediate supplied from the first microreactor R1 and an electrophile; and
a second microreactor R2 which receives the second mixture from the second micromixer M2 and reacts it to synthesize a compound represented by structural formula 4 or a compound represented by structural formula 4′. 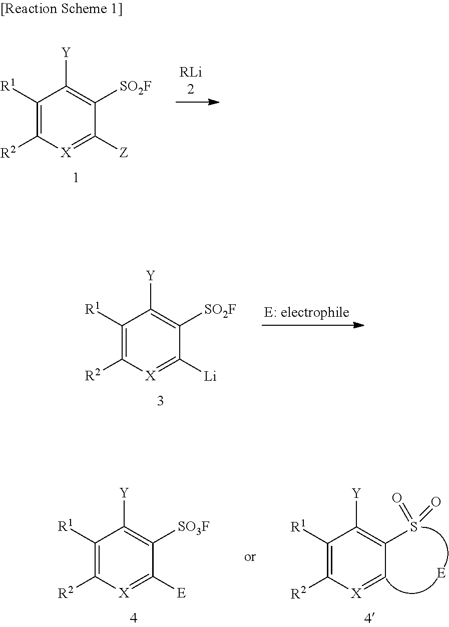
in the reaction scheme 1, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group,
X is a carbon atom or a nitrogen atom,
Y is a hydrogen atom, a fluorine atom, a chlorine atom, a bromine atom, or an iodine atom,
Z is a fluorine atom, a chlorine atom, a bromine atom, or an iodine atom,
R is C1 to C9 alkyl group of or C6 to C14 aryl group, and
E is an electrophile.

14. The ultra-fast synthesis apparatus of claim 13, wherein the reaction in the first microreactor R1 is carried out for a duration of 0.016 to 6.3 seconds.

15. The ultra-fast synthesis apparatus of claim 13, wherein the synthesis reaction in the second microreactor R2 is carried out for a duration of 1.5 to 3 seconds.

16. The ultra-fast synthesis apparatus of claim 13, wherein the reaction in the first microreactor R1 and in the second microreactor R2 are carried out at a temperature in a range of −58 to 25° C. respectively.

17. The ultra-fast synthesis apparatus of claim 13, wherein the ultra-fast synthesis apparatus further comprises:
a third micromixer M3 which prepares a third mixture by mixing a compound represented by structural formula 4 and a compound represented by structural formula 5 in reaction scheme 2; and
a third microreactor R3 which reacts the third mixture supplied from the third micromixer M3 to synthesize a compound represented by structural formula 6. 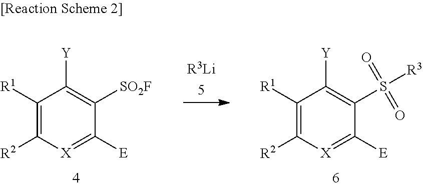
in the reaction scheme 2, R¹ and R² are identical to or different from each other, and are each independently a hydrogen atom, a substituted or unsubstituted C1 to C30 alkyl group, a substituted or unsubstituted C3 to C30 cycloalkyl group, a substituted or unsubstituted C1 to C30 heterocycloalkyl group, a substituted or unsubstituted C6 to C30 aryl group, or a substituted or unsubstituted C1 to C30 heteroaryl group, or at least one of R¹ and R² is further coupled with a carbon atom adjacent to a carbon atom linked therewith to form a substituted or unsubstituted fused C6 to C30 aryl group, or a substituted or unsubstituted fused C1 to C30 heteroaryl group,
X is a carbon atom or a nitrogen atom,
Y is a hydrogen atom, a fluorine atom, a chlorine atom, a bromine atom, or an iodine atom,
R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and
E is an electrophile.

18. The ultra-fast synthesis apparatus of claim 17, wherein the reaction in the third microreactor R3 is carried out for a duration of 5 to 10 seconds.

19. The ultra-fast synthesis apparatus of claim 13, wherein ultra-fast synthesis apparatus further comprises:
a fourth micromixer M4 which prepares a fourth mixture by mixing a compound represented by structural formula 5a and a compound represented by structural formula 5b in reaction scheme 3; and
a fourth microreactor R4 that reacts the fourth mixture supplied from the fourth micromixer M4 to synthesize a compound represented by structural formula 5. 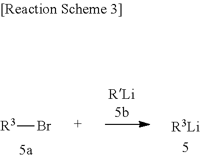
in the reaction scheme 3, R³ is a substituted or non-substituted C1 to C30 alkyl group, a substituted or non-substituted C3 to C30 cycloalkyl group, a substituted or non-substituted C1 to C30 heterocycloalkyl group, a substituted or non-substituted C6 to C30 aryl group, or a substituted or non-substituted C1 to C30 heteroalyl group, and
R′ is C1 to C9 alkyl group or C6 to C14 aryl group.

20. The ultra-fast synthesis apparatus of claim 19, wherein the reaction in the fourth microreactor R4 is carried out for a duration of 0.016 to 6.3 seconds.
